(19) United States
(12) Patent Application Publication
(10) Pub. No.: US 2026/0174993 A1
(43) Pub. Date: Jun. 25, 2026

(54) AGENT DELIVERY CATHETER WITH MICROPORE DESIGN

(71) Applicant: Precision NeuroMed Inc., Redwood City, CA (US)

(72) Inventor: Sandeep KUNWAR, Redwood City, CA (US)

(21) Appl. No.: 19/426,793

(22) Filed: Dec. 19, 2025

Related U.S. Application Data
(60) Provisional application No. 63/737,611, filed on Dec. 20, 2024.

(51) Int. Cl. A61M 25/00; A61M 5/172; A61M 25/01

(52) U.S. Cl. CPC A61M 25/007; A61M 5/1723; A61M 25/003; A61M 25/0054; A61M 25/0102; A61M 25/0113; A61M 2025/0002; A61M 2205/3303; A61M 2210/0693; A61M 2230/50; A61M 2230/65

(57) ABSTRACT

A catheter is disclosed that improves control over the shape and extent of agent distribution within biological tissue. The catheter includes a multi-lumen body with a sealed stylet lumen and an agent-delivery lumen terminating in an exposed distal tip portion having a plurality of micropores. Unlike end-port catheters, the micropores provide distributed outflow paths whose hydraulic resistance dominates over heterogeneous tissue resistance, reducing reflux and mitigating leakage into low-pressure structures. The distal tip portion may include multiple axial regions with differing pore densities, pore sizes, or no-pore gaps to tailor infusion geometry, and may be extendable or retractable to selectively expose regions. Additional embodiments may include zonal infusion with independent pressurization, dual-layer pore alignment to vary effective pore size, sensor-based closed-loop control, and microrobot-assisted navigation along straight or curved paths.

### CROSS-REFERENCE TO RELATED APPLICATION

This application claims the benefit of U.S. Provisional Application Ser. No. 63/737,611, filed Dec. 20, 2024, the entire content of which is incorporated by reference herein.

### TECHNICAL FIELD

Embodiments of the invention generally relate to convection-enhanced delivery (CED) therapy, and more particularly to a catheter which is used within the framework of CED therapy.

### BACKGROUND

Convection-enhanced delivery (CED) is a promising technique for delivering therapeutic agents directly to brain tissue. It bypasses the blood-brain barrier, a delicate defense system that restricts the passage of large molecules from the bloodstream into the brain. CED works by infusing drugs through a catheter placed within the brain parenchyma. A pressure gradient is then established, driving the therapeutic agent through the interstitial fluid, bathing a larger volume of tissue compared to traditional diffusion-based methods, with significantly higher concentration of drug.

Despite its advantages, CED faces challenges. Current single-port catheters can limit the distribution of the therapeutic agent, potentially leaving areas of the target tissue untreated. Additionally, achieving a uniform distribution throughout larger tumors or complex anatomical regions can be difficult. Furthermore, air bubbles within the catheter lumen can be a significant concern. Air bubbles can disrupt the flow of the therapeutic agent, leading to uneven distribution and potentially compromising treatment efficacy.

Another major limitation of conventional CED catheters is reflux. Reflux occurs when the infused therapeutic agent fluid flows out of the catheter tip and backs up along the external surface of the catheter, hindering its penetration into the target brain tissue. Reflux can be caused by several factors, including catheter design. Simple, single-port designs may not effectively prevent backflow (reflux), especially during pressure fluctuations or patient movement. Reflux not only reduces treatment efficacy by limiting drug delivery but can also lead to unintended exposure of healthy brain tissue to the therapeutic agent, potentially causing side effects.

Also, optimal CED catheters need to have small radius but be able to target deep tissue structures. Conventional CED catheters developed to overcome this often necessitate patients to be under general anesthesia in an operating room during the infusion process, due to their rigidity and potential for causing discomfort, or possibly under an MRI (magnetic resonance imaging) scanner for the whole duration of the treatment. This adds complexity and cost to the procedure and may not be suitable for all patients. There is a need for improved catheter designs that address these and other issues.

### SUMMARY

In one or more embodiments, a medical catheter includes a multi-lumen tube comprising a proximal end, a distal end, a first lumen, and a second lumen that is independent of the first lumen. The first lumen is sealed at the distal end. The catheter further includes a retractable (removable) stylet carried in the first lumen of the multi-lumen tube. The stylet is adapted to stiffen the multi-lumen tube during a placement process and is movable relative to the multi-lumen tube. The first lumen being sealed at the distal end prevents the retractable stylet from protruding from the distal end of the multi-lumen tube. The first lumen being sealed forms a hermetic barrier that vents only proximally through a hub of the catheter and limits entrainable gas volume to microliter order under high pressure (e.g., entrainable gas volume is limited to <0.5 microliters under, e.g., 300 mm Hg). The catheter further includes a capillary tube in the second lumen of the multi-lumen tube. The capillary tube includes a distal tip for releasing an agent at the distal tip to a biological tissue. The distal tip of the capillary tube is an exposed portion of the capillary tube that protrudes beyond the distal end of the multi-lumen tube.

In one or more embodiments, a catheter includes a tube comprising a proximal end, a distal end, and a lumen. The lumen is sealed at the distal end. The catheter further includes a retractable stylet carried in the lumen, wherein the retractable stylet is adapted to stiffen the tube during a placement process and is movable relative to the tube, wherein the lumen being sealed at the distal end prevents the retractable stylet from protruding from the distal end of the tube. The catheter further includes a coupler connected to the distal end of the tube. And the catheter further includes a capillary tube carried in the lumen, the capillary tube comprising a distal tip for releasing an agent at the distal tip to a biological tissue, wherein the distal tip of the capillary tube is an exposed portion of the capillary tube that protrudes beyond the distal end of the tube and a distal end of the coupler.

In one or more embodiments, a convection-enhanced delivery (CED) method includes a plurality of steps. The steps include a step of placing a distal tip of a catheter at a target location in the brain. The catheter includes a multi-lumen tube comprising a proximal end, a distal end, a first lumen, and a second lumen that is independent of the first lumen, the first lumen being sealed at the distal end. The catheter further includes a retractable stylet carried in the first lumen of the multi-lumen tube, wherein the stylet is adapted to stiffen the multi-lumen tube during a placement process and is movable relative to the multi-lumen tube, wherein the first lumen being sealed at the distal end prevents the retractable stylet from protruding from the distal end of the multi-lumen tube. The catheter further includes a capillary tube of the second lumen of the multi-lumen tube, the capillary tube comprising the distal tip for releasing an agent at the distal tip to a biological tissue, wherein the distal tip of the capillary tube is an exposed portion of the capillary tube that protrudes beyond the distal end of the multi-lumen tube. The steps of the method further include a step of removing the retractable stylet at least partially from the first lumen to impart flexibility to the catheter, and a step of delivering an agent through the capillary tube to the target location. In one or more embodiments, the catheter may further include a third lumen containing one or more sensors, such as sensors configured to detect reflux, distribution of the therapeutic agent, infusion pressure, temperature, catheter location, electrical impedance, or chemical concentration. Data from the sensor(s) may be transmitted to a control unit that automatically adjusts infusion parameters to optimize drug delivery and minimize reflux. In another embodiment, a method of convection-enhanced delivery includes placing the catheter at a target location, retracting the stylet to impart flexibility, delivering the agent through the capillary tube, and maintaining a seal between the catheter and brain tissue to reduce reflux during infusion. The method may further include detecting infusion parameters via the sensor(s), adjusting infusion parameters based on the detected data, and performing infusion under stereotactic guidance and conscious sedation. While the brain is provided as an example of target tissue with which this catheter may be used, the catheter may also be used to infuse material to or into other tissues, such as in the cardiac region, the gastrointestinal region, among others.

### BRIEF DESCRIPTION OF DRAWINGS

The disclosed embodiments have other advantages and features which will be more readily apparent from the detailed description, the appended claims, and the accompanying figures (or drawings). A brief introduction of the figures is below.

FIG. 1A is a schematic diagram showing a process of placing a conventional catheter in brain tissue.

FIG. 1B is a schematic diagram showing reflux during a process of infusing a drug in brain tissue with a conventional catheter.

FIG. 1C is a schematic diagram showing reflux during a process of infusing a drug in brain tissue with a conventional catheter.

FIG. 1D is a schematic diagram showing convection during a process of infusing a drug in brain tissue with a conventional catheter.

FIG. 2 is a schematic diagram showing a side view of a catheter for CED therapy, in accordance with one or more embodiments.

FIG. 3 is a schematic diagram showing a side view of a hub of a catheter for CED therapy, in accordance with one or more embodiments.

FIG. 4 is a schematic diagram showing a perspective view of a stylet after it is removed from a catheter for CED therapy, in accordance with one or more embodiments.

FIG. 5 is a schematic diagram showing a sectional view of a multi-lumen tube of a catheter for CED therapy, in accordance with one or more embodiments.

FIG. 6 is a schematic diagram showing a side view of a distal portion of a catheter for CED therapy, in accordance with one or more embodiments.

FIG. 7 is a schematic diagram showing a perspective view of a distal portion of a catheter for CED therapy, in accordance with one or more embodiments.

FIG. 8 is a schematic diagram showing a side view of a reinforcing member at a distal portion of a catheter for CED therapy, in accordance with one or more embodiments.

FIG. 9 is a schematic diagram showing a sectional view of a multi-lumen tube of a catheter for CED therapy, in accordance with one or more embodiments.

FIG. 10 is a flow chart illustrating a process for delivering a therapeutic agent to brain tissue, in accordance with one or more embodiments.

FIGS. 11 and 12 illustrate a micropore design for catheters, in accordance with some embodiments.

FIGS. 13A-13B and FIG. 14 illustrate the distal tip portion of a catheter with variable length, in accordance with some embodiments.

FIG. 15 illustrates zonal control and the micropores allowing for different shapes of agent distribution, in accordance with some embodiments.

FIGS. 16 and 17 are conceptual diagrams illustrating the theory of poor catheter design using the analogy of circuit current and resistance, in accordance with some embodiments.

FIG. 18 and FIG. 19 are conceptual diagrams illustrating a dual-layer catheter, in accordance with some embodiments.

FIG. 20 provides an example to illustrate the challenges in agent distribution, in accordance with some embodiments.

FIGS. 21 and 22 are conceptual diagrams illustrating a microrobot guiding the movement of the catheter, in accordance with some embodiments.

FIG. 23 is a flow chart illustrating a process for delivering a therapeutic agent to brain tissue using a microporous catheter, in accordance with one or more embodiments.

FIGS. 24A-24B illustrate exemplary alternate versions of the distal tip portion of the catheter, in accordance with some embodiments.

### DETAILED DESCRIPTION

The Figures (FIGS.) and the following description relate to preferred embodiments by way of illustration only. It should be noted that from the following discussion, alternative embodiments of the structures and methods disclosed herein will be readily recognized as viable alternatives that may be employed without departing from the principles of what is claimed.

Reference will now be made in detail to several embodiments, examples of which are illustrated in the accompanying figures. It is noted that wherever practicable similar or like reference numbers may be used in the figures and may indicate similar or like functionality. The figures depict embodiments of the disclosed system (or method) for purposes of illustration only. One skilled in the art will readily recognize from the following description that alternative embodiments of the structures and methods illustrated herein may be employed without departing from the principles described herein.

In the following description, for purposes of explanation, numerous specific details are set forth to provide a thorough understanding of the inventive concept. In the interest of clarity, not all features of an actual implementation are described. Moreover, the language used in this disclosure has been principally selected for readability and instructional purposes, and may not have been selected to delineate or circumscribe the inventive subject matter, resort to the claims being necessary to determine such inventive subject matter. Reference in this disclosure to “one embodiment” or to “an embodiment” or “another embodiment” means that a particular feature, structure, or characteristic described in connection with the embodiment is included in at least one embodiment of the invention, and multiple references to “one embodiment” or “an embodiment” or “another embodiment” should not be understood as necessarily all referring to the same embodiment.

The terms “a,” “an,” and “the” are not intended to refer to a singular entity unless explicitly so defined but include the general class of which a specific example may be used for illustration. The use of the terms “a” or “an” may therefore mean any number that is at least one, including “one,” “one or more,” “at least one,” and “one or more than one.” The term “or” means any of the alternatives and any combination of the alternatives, including all the alternatives, unless the alternatives are explicitly indicated as mutually exclusive. The phrase “at least one of” when combined with a list of items, means a single item from the list or any combination of items in the list. The phrase does not require all the listed items unless explicitly so defined.

### Configuration Overview

This disclosure pertains to a catheter that can be used for convection-enhanced delivery (CED) and that is designed to address limitations associated with conventional catheter designs. Techniques disclosed herein look to overcome challenges related to air bubbles, reflux, and the need for general anesthesia and MRI scanners during CED procedures. In one or more embodiments, the disclosed catheter is an invasive catheter that features a multi-lumen design, incorporating two primary lumens: a stylet lumen and an agent delivery lumen. In one or more embodiments, the disclosed catheter is an invasive catheter that features a multi-lumen design, incorporating three or more primary lumens: a stylet lumen, an agent delivery lumen, and one or more lumens to carry additional elements such as sensors, waveguides, or electrodes to the distal end. The term “multi-lumen” refers to a design with two or more lumens, though the design can include at least three lumens or at least four lumens or at least five lumens or more.

The stylet lumen houses a retractable stylet, providing rigidity during insertion into a subject's tissue, such as the brain parenchyma. In one or more embodiments, the stylet lumen houses the stylet and is sealed at a distal portion of the multi-lumen tube. As a result, when the stylet is removed from the lumen, the distal seal prevents any air bubbles from migrating into the biological tissue. In one or more embodiments, the stylet lumen is dedicated to the stylet and is not in fluid communication with other lumens. In some embodiments, the stylet is not sealed at the distal portion.

In one or more embodiments, the agent-delivery lumen is used for the delivery of an agent. The agent may be a therapeutic agent (e.g., drug), a pharmaceutical composition, a nutritional composition, and the like. In one or more embodiments, the agent-delivery lumen is dedicated for agent delivery and is not in fluid communication with other lumens. In one or more embodiments, the agent-delivery lumen may include a capillary tube that extends continuously from the proximal end or Luer lock adapter to the distal tip of the catheter. The distal portion of the catheter may include a coupler connected to a distal end of the multi-lumen tube created of a different or same material for contact with tissue and delivery of agent.

The catheter may gradually reduce in diameter towards the distal tip of the catheter so that the area at which the agent enters the biological tissue is minimized. The gradual change in diameter may take the form of stepped shape that steps downward in size towards the distal tip, tapering shape that tapers downward in size towards the distal tip, and/or any suitable combination. By way of example, the catheter may take the shape of multiple steps that gradually reduce the diameter(s) of the catheter towards the distal tip and minimize reflux. A first step at the distal tip of the capillary tube may be defined between the capillary tube and a reinforcement sleeve that may be coated with hydrophilic material (e.g., hydrogel) to improve a seal with brain tissue and minimize reflux. The reinforcement sleeve may be made of metal (e.g., magnetic resonance imaging-compatible metal) to impart rigidity to the distal tip of catheter during insertion, or it may be made of another rigid or substantially rigid material.

A second step further upstream from the first step may be defined between the reinforcement sleeve and a coupler (distal adapter) that connects the reinforcement sleeve with the multi-lumen tube. The stylet lumen may be designed such that when housing the stylet, the stylet may be inserted furthermost to a position that is proximal to and upstream of the coupler. As a result, since the stylet lumen is separate from the agent-delivery lumen and since the stylet lumen is sealed off upstream of the coupler, air that may be present in the stylet lumen cannot migrate past the coupler and into the brain tissue. The step design of the catheter and the coating on the reinforcement sleeve help minimize the risk of reflux and improve targeted drug delivery.

Upon reaching the target location within the tissue, the stylet can be withdrawn from the stylet lumen. This renders the catheter flexible, conforming to the tissue's natural contours (e.g., brain) and minimizing the potential for tissue damage during manipulation or drug infusion. The ability to remove the stylet eliminates the need for general anesthesia, potentially allowing for CED procedures under conscious sedation or local anesthesia, improving the subject's comfort and accessibility. The catheter may be secured to tissue at the skull (e.g., abone screw) or skin to prevent migration and easy removal after treatment.

Overall, the present disclosure provides a CED catheter that addresses critical limitations of conventional designs. The multi-lumen configuration with a sealed stylet lumen prevents air bubble formation, the step design starting from the distal tip of the catheter and surface coating (or other mechanism like an expansile outer member) minimizes reflux, and the removable stylet enhances flexibility during infusion, potentially reducing the need for general anesthesia, allowing patient to be mobile during infusion and allowing infusion outside of the operating room, while patient is awake. These advancements contribute to a safer, more efficient, and potentially less invasive CED procedure.

Also, in conventional CED systems, drug dispersal at the distal tip of the catheter may be dictated by the surrounding tissue's resistance; once infusate finds a low-pressure path, most of the flow may follow that route, causing reflux along the catheter body or leakage into adjacent vasculature and producing asymmetric, unpredictable drug distribution. This disclosure recognizes the need for controlling hydraulic resistance at the catheter-tissue interface for uniform delivery or achieving delivery of a desired shape or volume. In order to overcome the reflux or leakage problems of conventional CED systems, in some embodiments, a distal tip portion of the catheter according to the present disclosure may be configured as a microporous infusion head rather than a single outlet. For example, a distal end of a capillary tube of the CED catheter may include a plurality of micropores, from about 1 to 100 μm in diameter (e.g., 2 μm, 3 μm, 4 μm, 5 μm, 10 μm, 15 μm, 20 μm, 25 μm, 30 μm, 35 μm, 40 μm, 45 μm, 50 μm, 55 μm, 60 μm, 65 μm, 70 μm, 75 μm, 80 μm, 85 μm, 90 μm, 95 μm or any values or fractional values or sub-ranges within the range of 1 to 100 μm in diameter) and circumferentially arranged along a tubular tip that may be about 5-50 mm in length (e.g., 6 mm, 7 mm, 8 mm, 9 mm, 10 mm, 11 mm, 12 mm, 13 mm, 14 mm, 15 mm, 16 mm, 17 mm, 18 mm, 19 mm, 20 mm, 21 mm, 22 mm, 23 mm, 24 mm, 25 mm, 26 mm, 27 mm, 28 mm, 29 mm, 30 mm, 31 mm, 32 mm, 33 mm, 34 mm, 35 mm, 36 mm, 37 mm, 38 mm, 39 mm, 40 mm, 41 mm, 42 mm, 43 mm, 44 mm, 45 mm, 46 mm, 47 mm, 48 mm, 49 mm or any values or fractional values or sub-ranges within 5 to 50 mm. In some examples, the tip may be less than 30 mm in length or 1 mm to 30 mm in length, or it may be from 30-50 mm in length. It may also be above 50 mm, such as up to 75 or 100 mm or 200 mm, or any value, fractional value, or range within these numbers.

The microporous infusion head according to the present disclosure may create uniform or zonally targeted hydraulic resistance so that flow may be dictated by the pores rather than the heterogeneity of the tissue. By equalizing or adjusting pore resistance, the system according to the present disclosure may mitigate the problem of the path of least resistance of conventional CED systems. For example, with the microporous infusion head, even if one pore encounters a low-pressure zone, other pores of the infusion head may remain pressurized and continue to infuse at their intended rate, thereby expanding the convection volume and reducing reflux.

In some embodiments, to tailor the volume and shape of the infusion cloud, the microporous tip portion may be divided into discrete zones with different pore densities or diameters and connected to independent micro-infusion lines. Techniques disclosed herein may look to selectively pressurize these zones, thereby allowing the clinician to program customized distribution profiles and compensate for variations in tissue compliance. In some embodiments, the distal tip portion may be retractable or extendable, exposing more or fewer discrete zones as needed. Other embodiments of the microporous infusion head may utilize a dual layer structure in which an inner tube with fixed-size pores may slide within an outer sleeve or tube having larger, variable-size apertures; by rotating or translating the dual layers relative to one another, pore size and flow rate can be modulated dynamically. In some embodiments, the CED catheter may include sensors integrated into additional lumens of the multi-lumen tube to provide a closed loop system that can feed back tissue pressure or drug concentration data so that infusion parameters can be adjusted in real time. The microporous catheter may also employ a stylet or a microrobot so that the catheter can be deployed along straight or curved paths. Collectively, embodiments of the micropore-based catheter may provide a technical solution for precise, reflux-free convection enhanced delivery in delicate neural tissue.

### Example Reflux and Convection with a Conventional Catheter

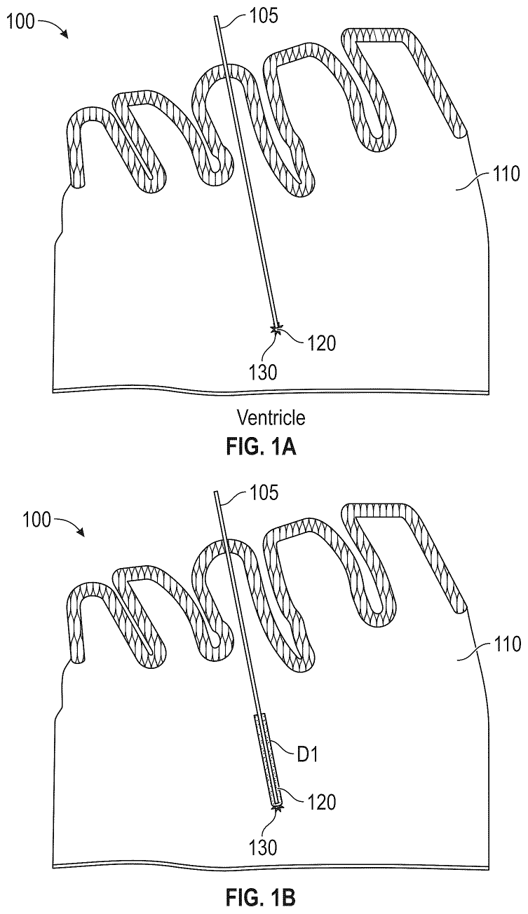

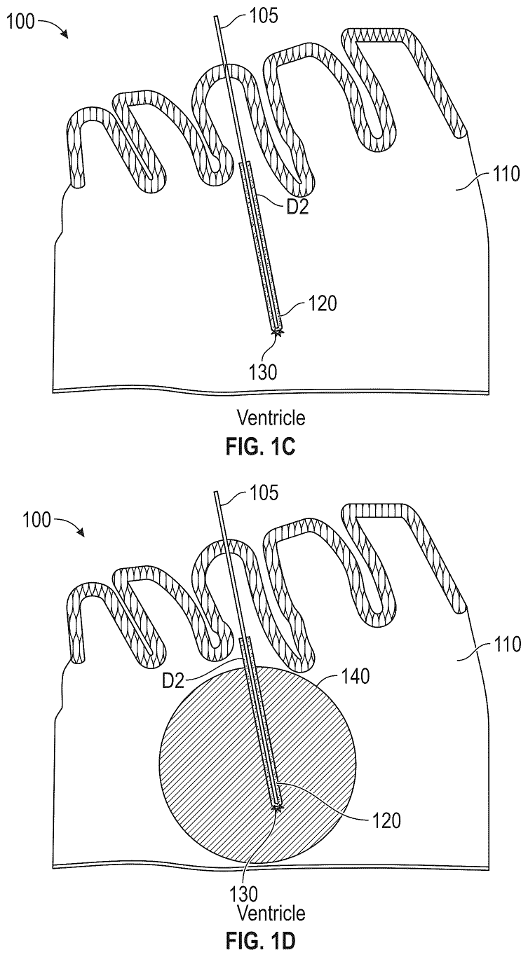

FIGS. 1A-1D illustrate the concepts of reflux and convection in CED therapy. FIG. 1A is a schematic diagram 100 showing a process of placing a conventional catheter 105 in brain tissue 110. Current stereotactic devices may be used to drill a hole and insert a bone screw into the skull. The bone screw defines a portal into the brain. The catheter 105 may be passed through the bone screw with seal and maneuvered to place a distal tip 120 of the catheter 105 at a target location 130 (e.g., 3-dimensional location having predetermined coordinates) in the brain tissue 110. Once the catheter 105 is positioned at the target location 130, the bone screw is tightened, which then secures the catheter 105 in place. After the placing process shown in FIG. 1A is complete, a process of infusing an agent (e.g., therapeutic agent) as shown in FIGS. 1B-1D may be started. While the brain tissue 110 is used as an example of a biological tissue, various embodiments of catheters described in this disclosure may be used for agent delivery to other suitable biological tissues.

FIGS. 1B-1C are schematic diagrams 100 showing reflux during a process of infusing an agent in the brain tissue 110 with the catheter 105. FIG. 1B shows that as the fluid carrying the therapeutic agent is infused from the catheter tip 120, the fluid leaks back out and up along the external surface of the catheter 105, instead of going outward into the brain tissue 110 (D1 in FIG. 1B). This is called reflux. Reflux occurs because the force required to push the fluid into the brain tissue, which is solid, may initially be greater than the force required to cause the fluid to flow up along the cavity created by the catheter 105 tube. Pressure is force per unit area, and as the area covered with the therapeutic fluid is built up and the reflux distance increases from D1 in FIG. 1B to D2 in FIG. 1C, the pressure around the catheter seal and the brain equilibrates. After reaching the equilibrium state shown in FIG. 1C, the therapeutic agent starts to convect as shown at 140 in FIG. 1D, go radially outward into the brain tissue 110. It is desirable to reduce the reflux distance D2 to improve treatment efficacy, avoid drug delivery to healthy brain tissue, and minimize loss of drug volume.

### Example Catheter for CED Therapy

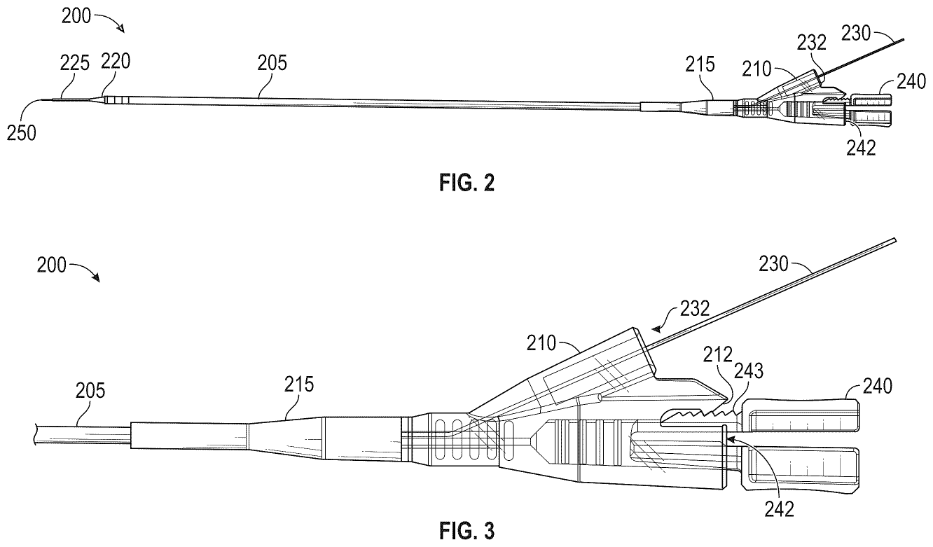

FIG. 2 is a schematic diagram showing a side view of a catheter 200 for CED therapy, in accordance with one or more embodiments. FIG. 3 is a schematic diagram showing a side view of a catheter hub 210 of the catheter 200, in accordance with one or more embodiments. FIGS. 2 and 3 show that the catheter 200 includes a multi-lumen tube 205, a catheter hub 210, a connecting member 215 (e.g., strain relief), a coupler 220, a reinforcement sleeve 225, a capillary tube 230, and a stylet 240. In other embodiments, the catheter 200 may exclude one or more of the components shown here. For example, the catheter 200 may exclude the reinforcement sleeve 225.

The multi-lumen tube 205 has a proximal end that is coupled to the catheter hub 210 via the connecting member 215. A distal end of the multi-lumen tube 205 is connected to the coupler 220. The multi-lumen tube 205 may be constructed from a biocompatible and flexible material. For example, the multi-lumen tube 205 may be constructed from polyamide (Pebax, Nylon), polyimide (PI), polyethylene (HDPE, LDPE), polyurethane or silicone, or from a polymer such as polyether ether ketone (PEEK) or a combination of these materials. A diameter of the multi-lumen tube 205 is narrow to minimize invasiveness while allowing sufficient internal space for the two or more lumens (e.g., agent-delivery lumen, stylet lumen) defined within. Although the illustrated embodiments show the multi-lumen tube 205 as having two lumens this is not intended to be limiting. In one or more embodiments, the multi-lumen tube 205 may define three or more lumens within a tube wall (e.g., sensor lumen in addition to agent-delivery lumen and stylet lumen).

In one or more embodiments, the total length of the multi-lumen tube 205 may be about in the range of about 1 and 16 inches (e.g., 2 inches, 3 inches, 4 inches, 5 inches, 6 inches, 7 inches, 8 inches, 9 inches, 10 inches, 11 inches, 12 inches, 13 inches, 14 inches, 15 inches, or any values or fractional values in between or any subranges in between 1 and 16 inches). In some cases, the tube 205 is longer, such as up to 20 inches, up to 25 inches or up to 30 inches. In one or more embodiments, the largest diameter of the multi-lumen tube 205 may be about in the range of about 500 and 5000 microns (e.g., 550 microns, 600 microns, 650 microns, 700 microns, 750 microns, 800 microns, 850 microns, 900 microns, 1000 microns, 1500 microns, 2000 microns, 2500 microns, 3000 microns, 3500 microns, 4000 microns, 4500 microns, or any values or fractional values in between or any subranges in between 500 and 5000 microns). For example, the total length of the multi-lumen tube 205 may be about 6 inches, and the largest diameter of the multi-lumen tube 205 may be about 1800 microns.

Although embodiments of the catheter 200 are described as including the multi-lumen tube 205 having two or more lumens, this may not always be the case. In one or more embodiments, the catheter 200 may include a tube which defines the main body of the catheter 200, where the tube defines a single-lumen through which the capillary tube 230 and the stylet 240 extend. For example, instead of having separate lumens for the capillary tube 230 and the stylet 240, the catheter 200 in accordance with one or more embodiments may include a single lumen through which at least the capillary tube 230, and the stylet 240 extend, where the stylet 240 is removable from the single lumen, and where the distal end of the single lumen tube terminates such that no air from the single lumen can escape out of the distal end of the catheter 200.

The connecting member 215 (e.g., strain relief) may be a sleeve or an adapter that attaches the multi-lumen tube 205 to the catheter hub 210. In one or more embodiments, the multi-lumen tube 205 is bonded to the catheter hub 210, and the connecting member 215 is a heat shrink that covers portions of both the multi-lumen tube 205 and the catheter hub. The connecting member illustrated has a few segments, including a cylindrical segment that is connected to and distal to the catheter hub. Connected to the cylindrical segment is a segment that tapers down to a narrower diameter in a conical shape. The tip of this tapered segment connects to another cylindrical segment that is distal to the conical-shaped form and connects to the multi-lumen tube 205. Some designs do not have all of these segments or have differently shaped segments. Alternately, the connecting member 215 is a molded component that covers a portion of the multi-lumen tube 205 and that snaps into the catheter hub 210. The catheter hub 210 serves as the external connection point for the catheter 200. The catheter hub 210 may include shafts that are in fluid communication with respective lumens of the multi-lumen tube 205. In the embodiment shown in FIGS. 2 and 3, the catheter hub 210 includes a first shaft 232 that is in fluid communication with an agent-delivery lumen of the multi-lumen tube 205, and a second shaft 242 that is in fluid communication with a stylet lumen of the multi-lumen tube 205. The catheter hub 210 may be made of a rigid, or semi-rigid, transparent material or may otherwise have a clear visualization window integrated into a hub wall, allowing for visual confirmation of, e.g., fluid flow within the agent-delivery lumen without presence of any air bubbles.

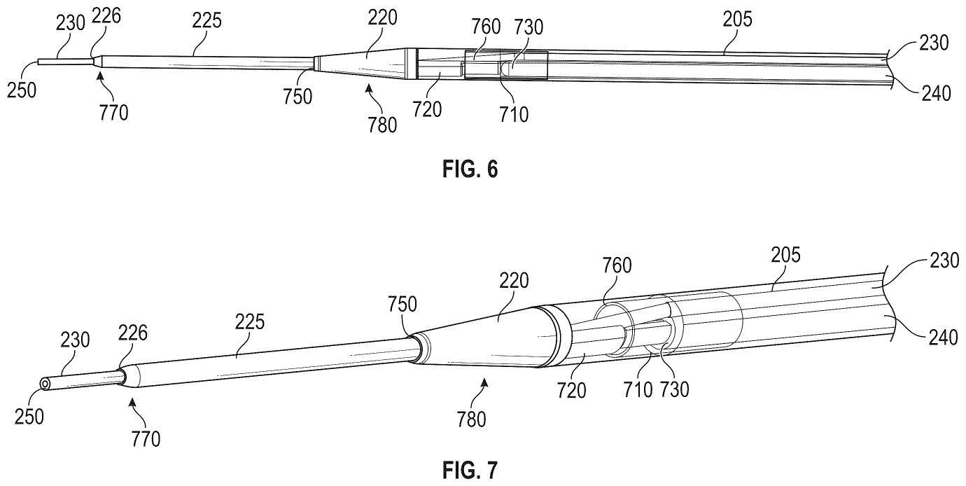

The coupler 220 may be connected to the distal end of the multi-lumen tube 205 and may limit a portion along a length of the catheter 200 through which the stylet 240 may extend. The reinforcement sleeve 225 may be connected to a distal end of the coupler 220. The coupler 220 has a conical shape and defines a step between the reinforcement sleeve 225 having a first diameter and the multi-lumen tube having a second diameter that is greater than the first diameter. For example, an outer diameter of the reinforcement sleeve may be in the range of about 0.1 to 0.2 millimeters, and an outer diameter of the multi-lumen tube may be in the range of about 1 to 3 millimeters. In one or more embodiments, an outer diameter of the reinforcement sleeve may be about 1 millimeters and an outer diameter of the multi-lumen tube may be about 3 millimeters. One some embodiments, the coupler 220 has a conical shape and may taper down to a tip that is connected to a proximal end of the reinforcement sleeve 225. Additional configuration details of the distal portion of the catheter 200 including the coupler 220 and the reinforcement sleeve 225 will be provided in connection with FIGS. 6-8. The step between the reinforcement sleeve 225 and the multi-lumen tube 205 may be a tapered portion, as shown in the drawings. Alternately, or in addition, the step may be an abrupt step (e.g., 90 degree angle) between the reinforcement sleeve 225 and the multi-lumen tube 205.

FIGS. 2 and 3 further show that the capillary tube 230 passes through the first shaft 232 of the catheter hub 210 and through a first lumen (agent-delivery lumen) of the multi-lumen tube 205, and the stylet 240 passes through the second shaft 242 of the catheter hub 210 and through a second lumen (stylet lumen) of the multi-lumen tube 205.

In one or more embodiments, the capillary tube 230 is a therapeutic agent delivery tube that is continuous from a Luer lock adapter (not shown) to a distal tip 250 of the catheter 200. The Luer lock adapter may be connected to an infusion pump (not shown) that pumps fluid including a target agent through the capillary tube 230 under pressure. A total length of the continuous capillary tube 230 from the distal tip 250 to the infusion pump may be in the order of several inches or several feet. For example, the total length may be in the range of about 2 inches to 10 feet (e.g., 5 inches, 10 inches, 15 inches, 20 inches, 2 feet, 3 feet, 4 feet, 5 feet, 6 feet, 7 feet, 8 feet, 9 feet, or any values or fractional values in between or any subranges in between).

The capillary tube 230 of the catheter 200 may be made of a material that is biocompatible and flexible. The material may be chosen such that it won't cause any adverse reactions or toxicity within the brain tissue. For example, the capillary tube 230 may be made of polyurethane that offers a good balance of flexibility and strength, making it suitable for navigating the brain without kinking or breaking. As another example, the capillary tube 230 may be made of silicone that is highly flexible and biocompatible, thereby minimizing tissue damage during insertion. As another example, the capillary tube 230 may be made of PEEK or a biocompatible plastic. In one or more embodiments, the capillary tube 230 may be made of fused silica, polyimide, silastic or any combination of suitable materials.

The flexibility may enable the capillary tube 230 to conform to brain contours, allowing for easy insertion of the flexible tip to a precise 3-dimensional location in the brain without causing excessive tissue damage. An outer diameter of the capillary tube 230 may be in the order of micrometers. Generally, the smaller the diameter of the capillary tube, the better the seal of the catheter with the surrounding tissue, resulting in better drug distribution and less reflux. As a result, it is advantageous to have a smaller diameter for the capillary tube 230. For example, the outer diameter of the capillary tube 230 may be in the range of about 100 micrometers to 2000 micrometers (e.g., 200, 300, 400, 500, 600, 700, 800, 900, 1000, 1500, micrometers, or any values or fractional values in between or an subranges in between 100 micrometers to 2000 micrometers). In one or more embodiments, the outer diameter of the capillary tube 230 at the distal tip may be about 500 micrometers.

The capillary tube 230 may be primed prior to or during insertion so that there is no void space or air bubbles in the agent-delivery lumen that may be pushed into the brain tissue during infusion. Since the diameter of the capillary tube 230 is small (e.g., about 500 micrometers) a total volume of the capillary tube 230 may be relatively small (e.g., about 6 microliters). As a result, dead volume is minimized and a very small amount of the drug is lost due to the priming process, which may be advantageous in certain types of treatments like gene therapy, where the cost of the drug may be very high. Priming the system prevents air bubbles from being introduced, and since the outer diameter of the capillary tube 230 is relatively small, the drug lost during the process is minimal. Another advantage of having the outer diameter of the capillary tube 230 in the stated range is a better seal between the outer surface of the catheter and the brain tissue, which reduces reflux (e.g., a shorter reflux distance) and improves convection. This in turn allows for increased pressure at the infusion pump, thereby increasing the infusion rate per minute and reducing the total infusion treatment time.

To provide rigidity for navigating the (flexible) distal tip 250 of the catheter 200 during insertion to a target location in the brain, the catheter 200 includes the stylet 240 that is accommodated in a dedicated stylet lumen of the multi-lumen tube 205 during insertion. The stylet 240 extends through a stylet lumen of the multi-lumen tube 205 and is adapted to stiffen the catheter 200 during the placing process. The stylet 240 is retractable (e.g., partially or entirely removable) for drug delivery, allowing the flexible multi-lumen tube 205 and the drug delivery tube 230 to conform to brain contours.

The stylet 240 may be made of a rigid material that imparts stiffness to the catheter 200 during insertion when the stylet 240 is accommodated in the stylet lumen of the multi-lumen tube 205. The stylet 240 material should be stiff enough to offer good control and stability while navigating the catheter 200 through brain tissue while preventing deflection, buckling or bending during insertion, ensuring an optimal insertion path for minimal tissue disruption. For example, the stylet 240 may be made of rigid or semi-rigid plastic, MRI-compatible metal or alloy, stainless steel, tungsten, titanium, PEEK, ceramic, glass and the like.

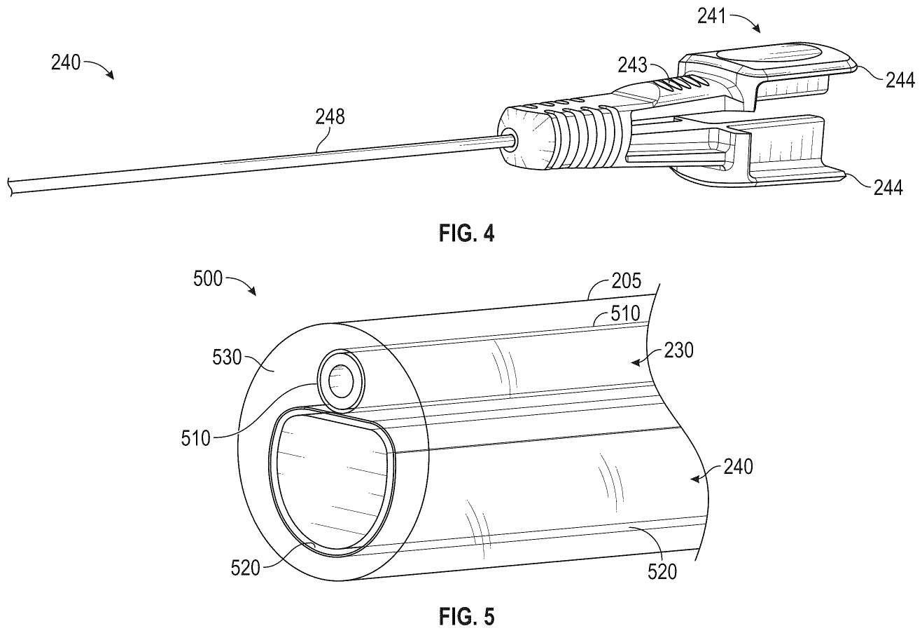

FIG. 4 shows the stylet 240 after it is removed from the catheter hub. As shown in FIGS. 2-4, the stylet 240 may have a handle 241 at a distal end and a main body 248 that is integrally formed with the handle 241. The handle 241 may include arms 244 having grooves 243 on an external surface that are adapted to engage with one or more ridges 212 formed in the stylet shaft 242 of the catheter hub 210. The arms 244 may be biased to remain engaged with the ridge 212 such that the stylet 240 remains locked in place in the stylet lumen of the multi-lumen tube 205 and in the stylet shaft 242 of the catheter hub 210 before and during the placing operation. An operator may compress the two arms 244 toward each other to release the engagement of the grooves 243 with the ridge 212, allowing the operator to either reposition the stylet 240 in the stylet lumen of the multi-lumen tube 205 or to remove the stylet 240 from the catheter 200 completely, as shown in FIG. 4. Other embodiments may employ a stylet having a different configuration for precisely placing the catheter 200 in target tissue. An outer diameter of the stylet 240 may be in the order of 1 mm. For example, the outer diameter of the stylet 240 may be in the range of about 0.5 millimeters to 3 millimeters (e.g., 0.75, 1, 1.5, 2, 2.5 millimeters, or any values or fractional values in between or an subranges in between 0.5 millimeters to 3 millimeters). In one or more embodiments, the outer diameter of the stylet 240 may be about 1.5 millimeters. A total length of the stylet may be in the range of about 25 millimeters to 500 millimeters (e.g., 30, 40, 50, 60, 70, 80, 90, 100, 150, 200, 250, 300, 350, 400, 450, or any values or fractional values in between or an subranges in between 25 millimeters to 500 millimeters). In one or more embodiments, an outer diameter of the stylet 240 may be about 0.1 inches.

FIG. 5 shows a sectional view 500 of the multi-lumen tube 205, in accordance with one or more embodiments. As shown in FIG. 5, the multi-lumen tube 205 defines two distinct lumens (channels or shafts) 510, 520 running longitudinally within tube wall 530. Although FIG. 5 shows the multi-lumen tube 205 as having two lumens 510, 520, this is not intended to be limiting. In one or more embodiments, the multi-lumen tube 205 may define three or more lumens within the tube wall 530.

In the embodiment shown in FIG. 5, the first lumen 510 is an agent-delivery lumen that houses the capillary tube 230 for delivering a therapeutic agent to a patient during drug infusion and the second lumen 520 is a stylet lumen for accommodating the stylet 240 to impart rigidity to the catheter 200 during the placing process. FIG. 5 shows that to minimize dead space, the inner diameter of the first lumen 510 is about the same as the outer diameter of the capillary tube 230, and the inner diameter of the second lumen 520 is about the same as the outer diameter of the stylet 240.

Although embodiments of the catheter 200 are described as including the agent-delivery lumen 510 and the capillary tube 230 are separate components wherein the capillary tube 230 is a continuous tube that passes through the lumen 510 of the multi-lumen tube 205, this may not always be the case. In one or more embodiments, the capillary tube 230 and the agent-delivery lumen 510 may be integrally formed into a single component. For example, the lumen 510 may itself define at least a portion of the capillary tube 230 such that there is no separate tube passing through the lumen 510. A tube may be coupled or integrally formed at the proximal and/or distal ends of the multi-lumen tube 205 to define one or both portions of the capillary tube 230 that extend from the proximal and/or distal ends of the multi-lumen tube 205.

FIGS. 6-8 are schematic views describing the configuration of a distal portion of the catheter 200, in accordance with one or more embodiments. In the embodiments shown in FIGS. 6-8, the distal portion of the catheter 200 includes at least the coupler 220, the reinforcement sleeve 225, and the distal tip 250 of the capillary tube 230.

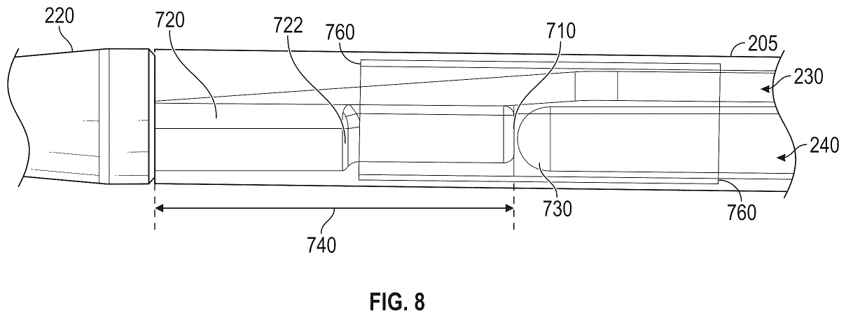

FIGS. 6-8 show that the multi-lumen tube 205 is attached to the coupler 220 at its distal end. The coupler 220 may be made of a rigid or semi-rigid material (e.g., plastic, PEEK, MRI-compatible metal, and the like). The distal end of the multi-lumen tube 205 and/or the coupler 220 act as a seal that prevent any air in the stylet lumen (520 in FIG. 5) escaping from the catheter 200 into brain tissue. That is, as shown most clearly in FIGS. 7 and 8, the stylet lumen may be sealed off within the tube wall (530 in FIG. 5) at a position 710 that is proximal to and upstream of the coupler 220. The stylet lumen 520 being sealed forms a hermetic barrier that vents only proximally through the catheter hub 210 and limits entrainable gas volume in the microliter order under high pressure (e.g., entrainable gas volume is limited to <0.5 microliters under, e.g., 300 mm Hg).

Although embodiments of the catheter 200 are described as including the coupler 220 that is a separate component attached to the multi-lumen tube 205, this may not always be the case. In one or more embodiments, the catheter 200 may include a tapered distal portion instead of the coupler 220, where the tapered distal portion is integrally formed with the multi-lumen tube 205.

FIGS. 6-8 also show that the coupler 220 includes a projecting member 720 that extends upstream of the coupler 220 and terminates at the sealed end of the stylet lumen at the position 710. The projecting member 720 may be adapted to reinforce the sealed end of the stylet lumen and prevent the distal end 730 of the stylet 240 from advancing any further in the stylet lumen of the multi-lumen tube 205 when the operator is pushing on the stylet 240 and the catheter 200 during the placing process. The stylet lumen may allow exit of air in the lumen from the catheter hub 210 at the proximal end when the stylet 240 is pushed into the lumen. That is, the stylet 240 may extend into the stylet lumen toward the coupler 220 until the distal end 730 of the stylet 240 contacts the projecting member 720 at the position 710 and cannot extend any further into the multi-lumen tube 205. Any air in the stylet lumen displaced by the advancing distal end 730 of the stylet 240 is pushed back toward the proximal end of the catheter 200 and out of the stylet shaft 242 of the catheter hub 210. Any air in the stylet lumen is thus prevented from being forced out into the tissue since the stylet lumen is sealed off within the tube walls and does not have any air outlet other than the opening of the stylet shaft 242 of the catheter hub 210 at the proximal end.

A portion 740 of the multi-lumen tube 205 downstream of the dead end of the stylet lumen at the position 710 may transition from a tube having multiple lumens (510, 520 in FIG. 5) into a tube having a single lumen within the tube wall, the single lumen corresponding to an agent-delivery lumen through which the capillary tube 230 may extend. As shown in FIGS. 6-8, the agent-delivery lumen in the portion 740 may extend at an angle to make the lumen and the capillary tube 230 within the lumen coaxial with the coupler 220 at a distal end 750 of the coupler 220.

FIGS. 6-8 also show that the tube wall at the distal portion of the multi-lumen tube 205 may include a reinforcing member 760. The reinforcing member 760 may be a cylindrical member that is adapted to stiffen the portion 740 and a region that overlaps with the distal end 730 of the stylet 240 when the distal end 730 is positioned in contact with the projecting member 720. For example, the reinforcing member 760 may be made of a metal such as titanium or a thermoplastic. Any material or composite that is suitable for medical use and has the appropriate properties (e.g., stiffness) may be used for manufacturing the reinforcing member 760. The reinforcing member 760 may be designed to prevent buckling and kinking at the position 710 where the distal tip 730 of the stylet 240 is positioned.

As shown most clearly in FIG. 8, the reinforcing member 760 may overlap at least partially with the projecting member 720 and also overlap at least partially with the distal end 730 of the stylet 240. The projecting member 720 may have a groove 722 to axially accommodate the cylindrical reinforcing member 760 while outer dimensions of the multi-lumen tube 205 remain unchanged. Since the stylet 240 terminates at the position 710 in the multi-lumen tube 205, which is more flexible than the stylet 240, the reinforcing member 760 imparts the necessary rigidity to ensure the distal portion of the catheter 200 has a desired stiffness gradient that is suitable for inserting and maneuvering the catheter 200 to a desired location within the brain with precision.

In one or more embodiments, as shown in FIGS. 6-7, the distal portion of the catheter 200 also includes the reinforcement sleeve 225. The reinforcement sleeve 225 is connected to the distal end 750 of the coupler 220. In some embodiments, the reinforcement sleeve 225 and the coupler 220 may be formed integrally. In other embodiments, the reinforcement sleeve 225 may be attached to the distal end 750 of the coupler 220 to form an airtight seal. In yet other embodiments, the reinforcement sleeve 225 may be omitted and the properties of the underlying capillary tube 230 may be modified to have the desired stiffness gradient imparted by the combination of the reinforcement sleeve 225 with the distal tip 250 axially extending from it. The reinforcement sleeve 225 may be made of any material that imparts stiffness to minimize deflection during the placing process. For example, the reinforcement sleeve 225 may be a metallic tube. As another example, the reinforcement sleeve 225 may be made of a thermoplastic or PEEK. Other similar materials or composites may also be used.

The reinforcement sleeve 225 is sealed and mated with the distal end of the agent-delivery lumen (510 in FIG. 5) of the multi-lumen tube 205 and extends the agent-delivery lumen distal to the coupler 220. The capillary tube 230 may extend though the agent-delivery lumen in the multi-lumen tube 205, in the coupler 220 and in the reinforcement sleeve 225 as a continuous tube. The reinforcement sleeve 225 surrounds a portion of the capillary tube 230 that protrudes beyond the distal end 750 of the coupler 220. That is, a portion including the distal tip 250 of the capillary tube 230 may protrude from a distal end 226 of the reinforcement sleeve 225 by a predetermined distance. The predetermined distance may be the range of about 1 millimeter to 50 millimeters. In one or more embodiments, the predetermined distance by which the capillary tube 230 extends from the distal end 226 of the reinforcement sleeve 225 to the distal tip 250 may be about 10 millimeters. The reinforcement sleeve 225 at the distal end 226 may be fused to the outer surface capillary tube 260 to create an airtight seal.

A length of the reinforcement sleeve 225 between the distal end 226 of the reinforcement sleeve 225 and the distal end 750 of the coupler 220 may be in the range of about 2 millimeters to 300 millimeters (e.g., 3, 5, 10, 20, 50, 100, 150, 200, 250 or any values or fractional values in between or an subranges in between 2 millimeters to 300 millimeters). In one or more embodiments, the length may be about 20 millimeters.

The reinforcement sleeve 225 may include a tapered portion 770 at the distal end 226. The tapered portion 770 defines a first step of dilation between the capillary tube 230 having an outer diameter that is less than an outer diameter of the reinforcement sleeve 225. During infusion, the drug flows out of the distal tip 250 of the catheter 200 and due to reflux, flows around and back against the outer surface of the capillary tube 230 until it reaches the tapered portion or step 770. The dilation at the tapered portion 770 helps create a better seal to the surrounding tissue, preventing the drug from flowing further back up against the outer surface of the reinforcement sleeve 225, thereby limiting the reflux distance to a length of the capillary tube 230 between the distal tip 250 and the distal end 226 of the reinforcement sleeve 225. Also, the infused fluid carrying the therapeutic agent hitting the tapered step 770 may create a turbulent flow that helps to accentuate the drug distribution by pushing the fluid outwards into the brain tissue and convect into the desired region of the brain. The step 770 may be a tapered portion, as shown in the drawings. Alternately, or in addition, the step may be an abrupt step (e.g., 90 degree angle) between the capillary tube 230 and the reinforcement sleeve 225.

FIGS. 6-8 also show that the coupler 220 has a tapered or conical shape that defines a second step 780 of dilation between the reinforcement sleeve 225 having an outer diameter that is less than an outer diameter of the multi-lumen tube 205. Similar to the first step of dilation between the capillary tube 230 and the reinforcement sleeve 225, the second step of dilation 780 also helps create a better seal to the surrounding tissue, preventing the drug from flowing back up against the outer surface of the guide tube 205, creating an additional backstop to prevent reflux and accentuate drug distribution into the brain tissue with a desired shape and in the desired area. Although FIGS. 6-7 illustrate an embodiment in which the catheter 200 has one step along the reinforcement sleeve 225, other embodiments may include two or more steps of successive larger dilations along a length of the catheter 200.

At least a portion of the outer surface of the reinforcement sleeve 225 may be treated with a hydrophilic coating to improve the seal with the brain tissue and further prevent reflux. Hydrophilic coatings such as hydrogel attract and absorb water. When applied to the catheter's 200 outer surface, the hydrogel coating swells upon contact with bodily fluids, creating a tighter and more secure seal between the catheter 200 and the surrounding tissue. This seal minimizes reflux of the infused drug, ensuring that the medication reaches its intended target. For example, at least a portion of the outer surface of the reinforcement sleeve 225 may be treated with a hydrogel-swell band. In some embodiments, the hydrogel-swell band may be made of a material that causes a 10-20% increase in its outside diameter within 60-90 seconds. The band may create a reflux threshold pressure that may be provided as input to a closed-loop system associated with the catheter 200 that may generate and regulate infusion pressure.

Mechanisms other than or in addition to hydrophilic coatings may also be employed to improve the seal and prevent reflux. Such mechanisms may define an expansile outer member that is adapted to have a first diameter during the placing process and a second diameter during an infusion process, the second diameter being greater than the first diameter. The expansile outer member may be utilized alternately or in addition to the reinforcement sleeve 225 for preventing reflux. In some embodiments, a shape morphism actuation of the expansile outer member may be at 32-35 degrees Celsius with saline concentration of 0.9% or the shape morphism actuation of the expansile outer member may be by the infusate. For example, shape memory polymers designed to change shape in response to body temperature can be used, potentially improving the seal as they conform to the tissue. Biocompatible adhesives (organic compounds, inorganic compounds) can be also used to create a stronger bond between the catheter and tissue. Mechanical mechanisms such as balloon catheters or expandable tip catheters may be employed as well. For example, the reinforcement sleeve 225 may have one or more balloons at the distal end 226 that can be inflated to create a secure seal. As another example, the reinforcement sleeve 225 may have an expandable tip at the distal end 226 that can be deployed to improve the seal.

In one or more embodiments, the catheter 200 may include one or more sensors to provide a closed self-sufficient system for controlled drug infusion. The sensors may be configured to measure signals at one or more positions along a length the catheter 200. Based on the measured sensor data, a control unit that may be included in the catheter (e.g., in the hub 210) or in equipment connected to the catheter (e.g., at the infusion pump) may detect various conditions related to catheter placement or drug infusion such as drug reflux, drug distribution, or treatment effect.

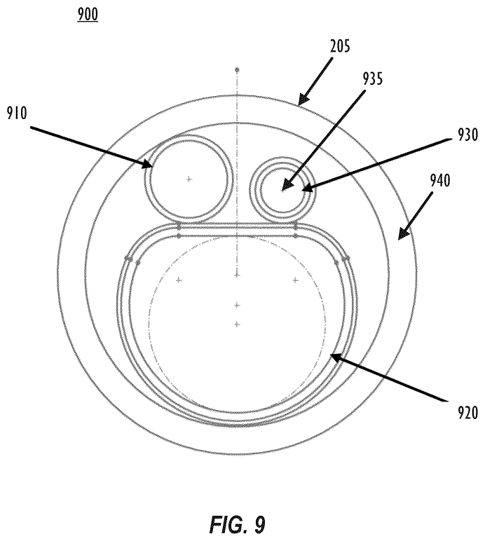

In one or more embodiments, the multi-lumen tube may include additional lumens (e.g., a third lumen (not shown) in addition to lumens 510, 520 within the tube wall 530 in FIG. 5) that may be dedicated to wiring or waveguides (e.g., fiber optic filament) for the sensor(s) and the catheter 200 may include components (e.g., electrodes) at various positions along the length of the catheter (e.g., at the reinforcement sleeve 225) to detect sensor data. For example, the sensor may be a pressure sensor configured to detect a pressure at the distal tip 250 or at the distal end 226 of the reinforcement sleeve 225, in-line, or at the infusion pump. The pressure sensor data may be used by a controller to adjust the infusion rate during the infusion process. For example, a controller may be able to detect at what pressure does the seal between the catheter 200 and the tissue finally break and drive the infusion rate as high as possible based on this information, thereby shortening the duration of the infusion treatment, or decreasing/halting infusion if a sudden change in pressure occurs. FIG. 9 illustrates a sectional view 900 of the multi-lumen tube 205, in accordance with one or more embodiments. As shown in FIG. 9, the multi-lumen tube 205 defines three distinct lumens (channels or shafts) 910 (e.g., for the capillary tube 230), 920 (e.g., for the stylet 240), and 930 running longitudinally within tube wall 940. Lumen 930 may include a pressure sensor 935 for monitoring pressure at, e.g., the distal tip of the catheter.

As another example, the sensor may be an electrical sensor to measure impedance or electrical parameters. In embodiments where the reinforcement sleeve 225 is a metallic tube, the electrical sensor may be implemented by carving out portions of the metallic tube and introduce one or more electrodes embedded within the reinforcement sleeve 225 such that they are flush with the external surface of the reinforcement sleeve. An impedance reading can then be obtained using the one or more electrodes in the reinforcement sleeve 225 and optionally, ground.

As yet another example, one or more electrodes may be placed on one or more surfaces at a distal end of the catheter. The electrodes may be placed as rings or as segments along the distal end and extending back along a portion or an entire length of the catheter. The electrodes may measure impedance, electrical activity, or chemical activity. The measurement may be utilized by a control system of the catheter to control one or more parameters during an infusion operation. Alternately, or in addition, the measurement data may be utilized after the infusion operation to determine characteristics of the infusion.

As yet another example, the sensor may be a positional sensor (e.g., coil) to measure deflection. As yet another example, the sensor may be a temperature sensor to measure a temperature at one or more positions along the catheter. Yet another example, the sensor may be a chemical or electrolyte sensor to detect a level of concentration of a chemical or electrolyte at one or more positions along the catheter. For example, the chemical sensor may be utilized to detect locations along the catheter where the drug is contacting the catheter to detect a reflux distance. Another example is a chemical sensor to monitor the therapeutic outcome of the infused agent. Yet another example, the sensor may be a positioning sensor (e.g., electromagnetic, sonographic) to determine the precise location of the distal tip of the catheter. For example, an electromagnetic position sensor (e.g., a position tracker manufactured by POLHEMUS INC.) may determine the precise location of the distal tip without the need for medical imaging like X-ray or MRI. Yet another example, an ultrasound transducer to image the tissue in the region outside the catheter.

In one or more embodiments, based on the sensor data, a controller may detect one or more properties during an infusion process, and control one or more parameters of the infusion process based on the detected one or more properties. For example, a controller at the infusion pump may dynamically control and maximize the infusion rate and when leakage or reflux is detected, manipulate the infusion rate to be able to slow down, get a better seal again, detect the improved seal, and then ramp the infusion rate back up again while continuously monitoring the sensor data.

In some embodiments, the catheter 200 may further comprise a third lumen (e.g., 930 in FIG. 9) that houses one or more sensors configured to provide real-time infusion data. The sensors may include one or more of: a reflux sensor, an agent distribution sensor, a pressure sensor, a temperature sensor, a positioning sensor, an electrical impedance sensor, or a chemical concentration sensor. Sensor outputs may be transmitted to a control unit that may be integrated into the infusion pump or into the catheter hub 210, to automatically adjust infusion parameters such as rate, pressure, or duration. In this way, the system may provide a closed-loop infusion control that reduces the likelihood of reflux and maximizes therapeutic efficiency.

In some embodiments, a method of using the catheter 200 may include placing the distal tip 250 under stereotactic guidance, retracting the stylet 240 from the stylet lumen 520 to impart flexibility, and delivering a therapeutic agent through the capillary tube 230 while maintaining a tissue seal (e.g., at the reinforcement sleeve 225). The infusion may be performed under conscious sedation, allowing patient comfort without requiring general anesthesia. Exemplary therapeutic agents include proteins, liposomal nanoparticles, viral or non-viral gene therapy vectors, chemotherapeutics, or combinations thereof. The stepwise diameter changes of the catheter 200, such as those defined between the capillary tube 230 and the reinforcement sleeve 225 (step 770), and between the reinforcement sleeve 225 and the multi-lumen tube 205 (step 780), together with respective hydrophilic coatings or expansile members, may limit reflux distance to less than 10 mm during infusion.

Prior to placement, the capillary tube 230 may be primed to minimize dead volume, for example to less than about 10 microliters, thereby conserving therapeutic material in high-value treatments such as gene therapy. During infusion, the control unit may use the measured sensor data from, e.g., a pressure sensor, electrodes embedded in the reinforcement sleeve 225, or other sensors positioned in third lumen, to enforce infusion rate limits, trigger alarms to alert the operator, or automatically modulate infusion to minimize drug loss while maximizing infusion efficiency.

### Example Method of Delivering a Therapeutic Agent to Brain Tissue

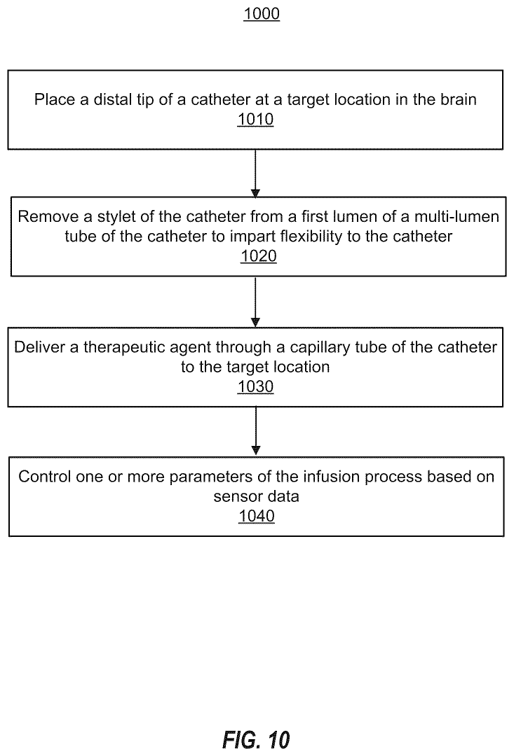

FIG. 10 is a flow chart illustrating a process 1000 for delivering a therapeutic agent to brain tissue, in accordance with one or more embodiments. A similar method may be used for other types of tissue.

The method 1000 begins with an operator placing 1010 a distal tip of a catheter (e.g., catheter 200 of FIGS. 2-9) at a target location in the brain. For example, as shown in FIGS. 2, 6, 7, since the catheter has a stiffness gradient such that the distal tip 250 is flexible while the reinforcement sleeve 225, the coupler 220, and the multi-lumen tube 205 with the reinforcing member 760 and the stylet 240 in a stylet lumen are more rigid, the operator can maneuver and guide the distal tip to a target location in the brain without deflection.

After guiding the distal tip to the target location, the operator may remove 1020 the stylet from the catheter (e.g., from lumen 510 in FIG. 5) to impart flexibility to the catheter. Since the stylet is removed, the multi-lumen tube now becomes relatively more flexible. As a result, the patient can remain more mobile and need not be in the operating room during subsequent drug infusion.

The operator may then deliver 1030 a therapeutic agent through a capillary tube (e.g., 230 in FIGS. 2, 3, 5-8) of the catheter to the target location. Since the catheter may include the step design with one or more steps (e.g., step 770 in FIG. 7), since an external surface of the catheter may be subject to, e.g., a hydrophilic coating at one or more locations, and/or since the catheter may include an expansile outer member, the catheter may prevent reflux by creating a seal between brain tissue and the outer surface of the catheter. As a result, the drug delivery can be precisely targeted to achieve a desired distribution with minimal reflux. Also, infusion rate can be increased without breaking the seal between catheter and tissue.

The catheter may further include one or more sensors (e.g., pressure sensor 935 of FIG. 9) and a control system may (manually, semi-automatically, or fully autonomously) control 1040 one or more parameters (e.g., infusion rate) of the infusion process based on sensor data (e.g., data indicating one or more of a reflux condition, a distribution condition, a brain density metric, an infusion location, and the like) to optimize the infusion process.

### Example Micropore Design

In convection enhanced delivery (CED) systems that employ a single discharge port at a distal tip of the infusion lumen, the hydrodynamics of the infusion may be governed by the surrounding interstitial pressure gradients. If the advancing bolus encounters a region with substantially lower hydrostatic pressure, such as a cerebrospinal fluid compartment, sulcal cleft, perivascular space, or large vessel, the infusate may preferentially divert into that sink. Thus, once a low-resistance conduit is breached, the bulk of the fluid may follow the path of least resistance, producing reflux along the catheter shaft or in the low-resistance zone and starving other areas of the tissue of therapeutic agent. This phenomenon may stall the expansion of the convection volume and can lead to asymmetric, unpredictable drug distribution or failure of the infusion. Even simple multipore designs may not adequately address the problem: when one pore opens into a low-pressure zone, it may dominate the flow field, effectively short circuiting the other ports and leaving other pores underutilized.

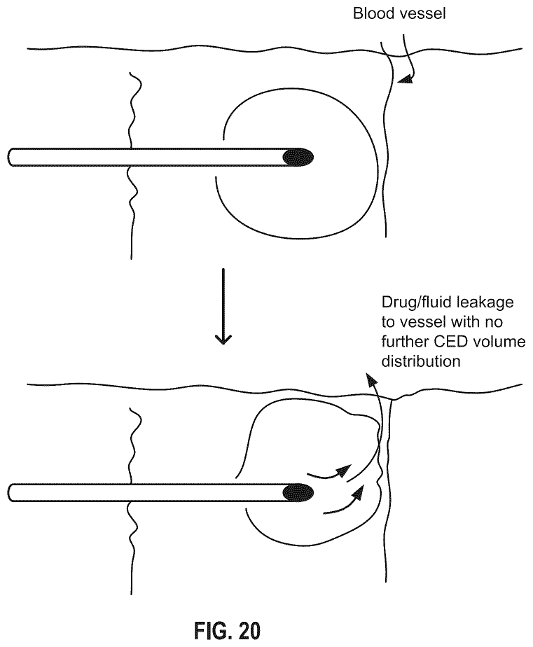

FIG. 20 schematically illustrates these limitations. At the top, the conceptual diagram illustrates a current single port or limited multipore catheter, where agent distribution (illustrated by the circle) is dependent on tissue pressure. That is, because the infusate flow relies on native tissue resistance to maintain backpressure, the shape and extent of the convection cloud is dictated by the spatial distribution of low-pressure zones. As depicted in the bottom panel of FIG. 20, when the catheter tip lies adjacent to a blood vessel, sulcus or other compliant low-pressure compartment, the infusate may rapidly leaks into that sink, as indicated by the arrow, and the infusion may fail to propagate further into the target tissue. That is, if the delivery is near a blood vessel or another low-pressure zone, the agent will leak to the vessel with no further CED volume distribution. The resulting distribution may be truncated and shifted toward the leak, leaving target regions untreated or undertreated. This example underscores the need for a catheter architecture that decouples flow resistance from tissue heterogeneity and prevents preferential leakage into low pressure conduits.

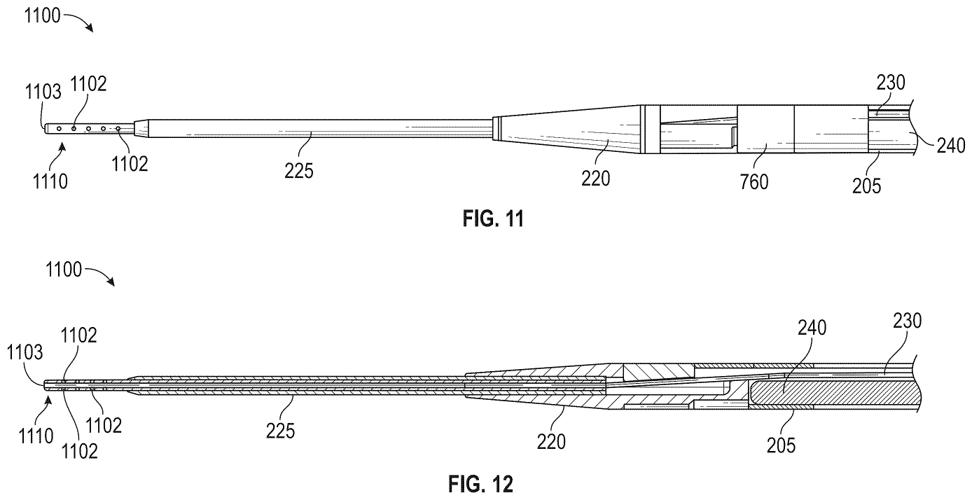

In some embodiments, the microporous infusion head described above may be implemented as illustrated in FIGS. 11 and 12. Except as noted herein, the catheter 1100 of FIGS. 11-12 may retain the general architecture of the multi-lumen catheter 200 described with respect to FIGS. 2-10, including a multi-lumen tube 205 having a first lumen that receives a retractable stylet 240 and a second, independent lumen for a capillary tube 230. The capillary tube 230 may terminate distally in a distal tip portion 1110 that extends beyond coupler 220 at the distal end of the multi-lumen tube 205. The distal tip portion 1110 may form a microporous infusion head that is formed on an exposed portion of the capillary tube 230 that extends beyond the reinforcement sleeve 225 and the coupler 220. As in other embodiments, the stylet 240 may extend through the first lumen to stiffen the catheter 1100 during placement, and the stylet 240 may terminate proximally of the coupler 220 such that the first lumen housing the stylet 240 remains sealed at its distal end to preclude air entrainment.

The distal tip portion 1110 may have a tubular structure having an outer surface provided with a plurality of micropores 1102. Each micropore 1102 may be formed by, e.g., by laser micromachining, etching, or other microfabrication technique and may have a hydraulic diameter in a range of about 1 μm to about 100 μm (e.g., 2, 5, 10, 20, 30, 40, 50, 60, 70, 80, 90 100 μm or any values or fractional values or subranges in between 1 μm to about 100 μm), and in some embodiments from about 10 μm to about 40 μm. Micropores 1102 may extend completely through the wall of the distal tip portion 1110 so that a lumen of the capillary tube 225 is in fluid communication with the biological tissue surrounding the distal tip portion 1110. In some embodiments, an additional design consideration involves the configuration of micropores 1102 at the distal tip 1103 of the catheter 1100. In some embodiments, the micropores 1102 may be circumferentially arrayed around the distal tip portion 1110 and may be distributed along a length of the distal tip portion 1110 with a constant or variable spacing. By selecting the number, size (e.g., diameter), and spacing of the micropores 1102, the fluidic resistance of the distal tip portion 1110 may be tuned so that micropores 1102 may govern flow rather than the flow being dictated by tissue heterogeneity, as described above. In some embodiments, micropores 1102 may be arranged into two or more discrete zones along the distal tip portion 1110, each zone having a different average pore size and/or pore density than an adjacent zone. In such embodiments, the second lumen of the multi-lumen tube 205, the coupler 220, and the reinforcement sleeve 225 may house separate micro-infusion channels (not shown) within the lumen or the wall of (or instead of) the capillary tube 230. Alternately, the capillary tube 230 may branch out into the separate micro-infusion channels at or distal to the coupler 220 or the reinforcement sleeve 225. The separate micro-infusion channels at the distal tip portion 1110 may then respectively fluidly couple to the different zones of micropores to provide independent and separately controllable infusion lines. One or more barriers (not shown) may be disposed in the distal tip portion 1110 between adjacent zones to fluidly isolate them from each other so that pressure in one zone does not equalize with pressure in another zone, thereby enabling independent control of infusion rate and volume for each zone. When larger and smaller micropores 1102 are used in adjacent zones, such barriers help prevent the larger pores from dominating the flow.

A length of the distal tip portion 1110 may be adjustable. In some embodiments, the catheter 1100 may be configured to permit the distal tip portion 1110 to extend or retract relative to the distal end of the multi-lumen tube 205 (e.g., relative to the distal end of the reinforcement sleeve 225 in embodiments where the sleeve 225 is provided, relative to the distal end of the coupler 220 in embodiments where the sleeve 225 is not provided, and the like). For example, the distal tip portion 1110 to extend or retract to adjust the length of the distal tip portion 1110 between a range of about 5 mm to about 30 mm. By extending the distal tip portion 1110 further from the multi-lumen tube 205, additional zones of micropores 1102 may be exposed, whereas retracting the distal tip portion 1110 may hide some of the zones of the micropores 1102, thereby tailoring the volume of distribution. The distal tip portion 1110 may be constructed to be rigid, transition from rigid to flexible, or be entirely flexible.

In some embodiments, micropores 1102 may be equipped with micro-valves or membranes that are selectively actuatable between open and closed states in response to electrical, mechanical, or thermal signals to further modulate flow. That is, by incorporating micro-valves or flexible membranes into individual micropores, the catheter 1100 can dynamically regulate which pores contribute to the infusion at any given moment. For example, each micro-valve may be opened or closed in response to control signals from a proximal controller, allowing certain zones of micropores to be active while others remain occluded, or modulating the degree of opening to fine-tune local flow resistance. In this manner, the overall flow distribution can be shaped in real time to match the geometry of the target tissue, compensate for detected pressure variations, or deliver different agents through different pore sets without physically repositioning the catheter.

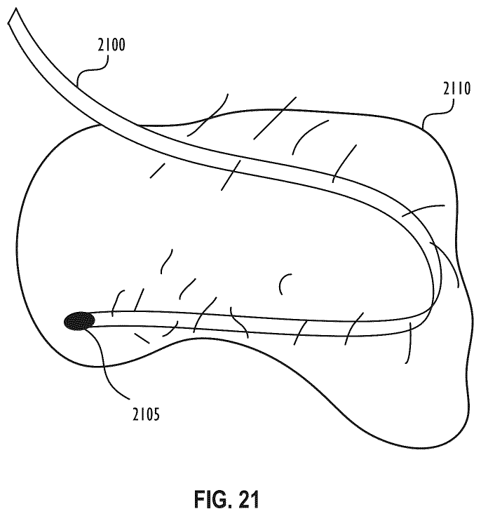

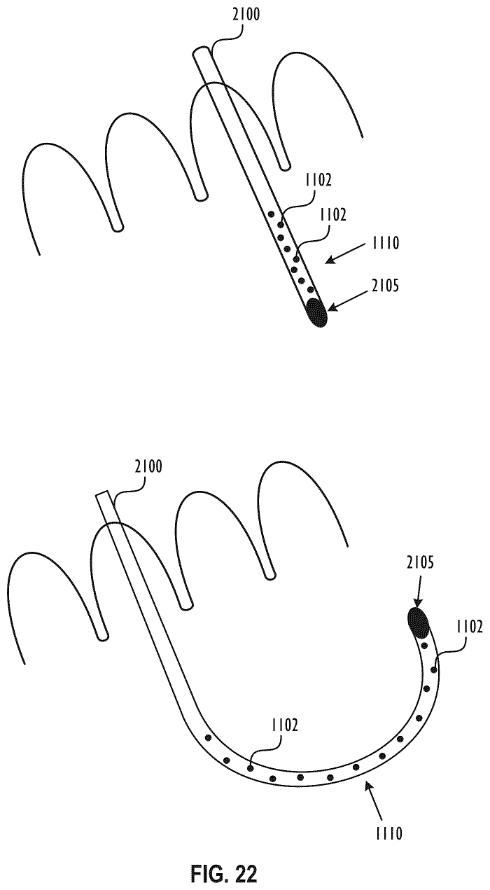

To support a closed loop control of infusion, the catheter 1100 may include one or more sensors (not shown) at or near the distal tip portion 1100. The sensors may be carried in one or more additional lumens of the multi-lumen tube 205 (e.g., as shown in FIG. 9) and may include pressure sensors, temperature sensors, chemical concentration sensors, electrical impedance sensors, reflux detectors, or imaging elements such as ultrasound transducers. Data from the sensor(s) may be supplied to a control unit (not shown) configured to adjust infusion parameters such as flow rate, infusion pressure, or zone selection in real time. The multi-lumen tube 205 may further house electrical leads or optical waveguides to supply power or control signals to the sensors and actuators at distal tip portion 1110. In some embodiments, a mechanical actuator or microrobot (FIGS. 21-22) may be coupled to the distal tip portion 1110 to assist in advancing the distal tip portion 1110 along a predetermined straight or curved trajectory within tissue. For example, the microrobot may pull or push the distal tip portion 1110 or may otherwise assist with fine grained navigation while the stylet 240 remains within the first lumen during placement, after which the stylet may be withdrawn to impart flexibility.

In some embodiments, the catheter 1100 with the micropores 1102 may enhance fluid distribution by creating a natural resistance to flow rate such that the primary resistance to flow may be determined by the micropores rather than by the surrounding tissue. As a result, if a micropore 1102 encounters a low-pressure path, the flow through the other micropores 1102 may remain unaffected, leading to improved drug distribution and convection volume. As a result, the catheter 1100 design may reduce the impact of tissue density variations and mitigate the effects of leakage, as tissue resistance does not significantly alter the pressure or flow through the other micropores.

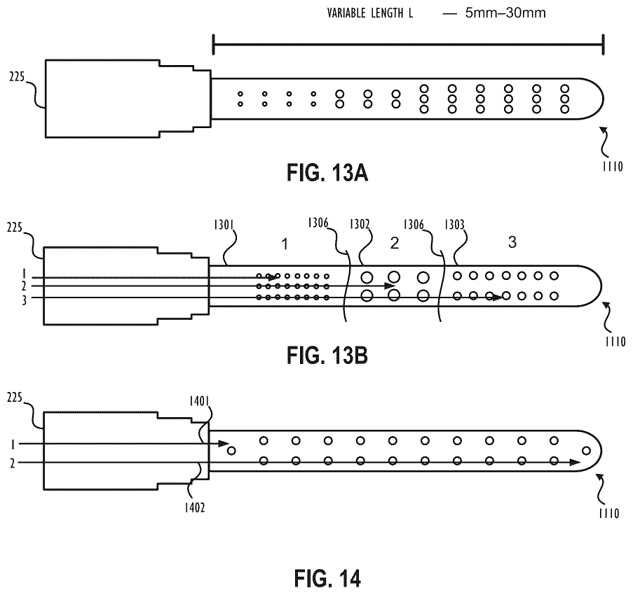

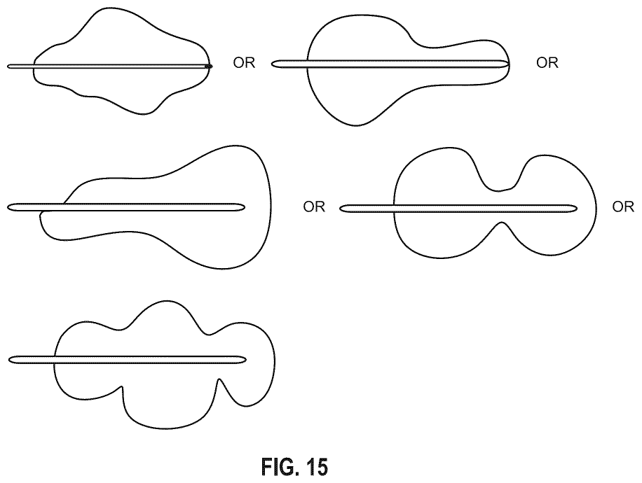

In some embodiments, and as depicted in FIGS. 13A-13B and 14, the distal tip portion 1110 of the capillary tube 230 can be configured as an axially extendable section that carries multiple zones of micropores 1102. The distal tip portion 1110 may be telescopically or otherwise slidably coupled to the remainder of the catheter (e.g., to sleeve 225), so that its exposed length L can be varied, for example, between about 5 mm and about 30 mm. In some embodiments, as illustrated in FIG. 13B, the distal tip portion 1110 may be divided into a plurality of zones, such as a first zone 1301 proximal to the reinforcement sleeve 225, a second zone 1302 adjacent the first zone, and a third zone 1303 distal to the second zone. Each zone may carry a distinct population of micropores 1102 whose average diameter and/or spacing may differ from that of an adjacent zone. For example, the first zone 1301 may contain a relatively high density of small-diameter micropores (e.g., on the order of 10-20 μm), the third zone 1303 may contain a medium density of medium-sized micropores (e.g., 20-40 μm), and the second zone 1302 may contain a lower density of larger-diameter micropores (e.g., 40-100 μm). The micropores may be formed circumferentially around the distal tip portion 1110 within each zone, and the arrangement of pore size, density and distribution within each zone may be selected to achieve predetermined infusion profiles.

By extending or retracting the distal tip portion 1110 relative to the distal end of the multi-lumen tube 205 (e.g., relative to sleeve 225), the operator may expose one or more zones 1301-1303 of micropores 1102 to the tissue while shielding the others within the reinforcement sleeve 225. This adjustability may allow the effective length of the microporous infusion head to be matched to the target volume, enabling a highly concentrated delivery when only the zone 1303 is exposed, or a broader, more diffuse distribution when additional zones 1302 and/or 1301 are sequentially exposed. Because each zone may be coupled to one or more independent micro-infusion channels (not shown) and separated by internal barriers (represented by 1306), infusion parameters such as pressure, flow rate or even therapeutic agent can be varied from zone to zone. The micro-infusion channels may be routed through the wall of the capillary tube 230 or provided as separate lumens in the distal adaptor 1120 such that each zone receives fluid from a dedicated infusion line. The barriers 1306 between zones 1301-1303 may prevent cross-flow and permit independent pressure control, thereby allowing the user to increase infusion pressure in one zone facing dense or high-resistance tissue while maintaining a lower pressure in a zones facing more compliant tissue (or where there is low pressure). The actuatable micropores described above can further be employed in the embodiments shown in FIGS. 13-14 to further refine infusion control by opening or closing selected pores within each zone in response to electrical, mechanical or thermal signals.

FIG. 14 shows an embodiment in which two infusion lines independently supply different sets of micropores. In the example of FIG. 14, there are two zones and the arrows indicate infusate flow to either the distal 1402 or the proximal 1401 end of the distal tip portion 1110. Through this architecture, different therapeutic agents or dosages may be delivered concurrently to distinct regions of the target tissue, or a single agent may be delivered with a spatially graded concentration.

FIG. 15 illustrates that, by varying the exposed length of the distal tip portion or by varying the infusion parameters on a per-zone basis, a wide variety of distribution shapes can be generated (based on tissue characteristics). For instance, selectively pressurizing a proximal zone results in a compact, lobular distribution localized around the catheter axis. Pressurizing a second distal zone while maintaining a first proximal zone at a lower pressure may produce an elongated distribution that spreads more distal to the insertion point. Concurrently infusing through both end zones while occluding a middle zone might yield a bilobed or “hourglass” distribution. For each zone, the catheter 1100 may include sensors, such as pressure or chemical sensors, to monitor local conditions and provide feedback to a control unit that can autonomously adjust the flow rate from each infusion line. Moreover, because the zones are independently addressable, different therapeutic agents or dosages can be delivered concurrently or sequentially through different zones, enabling a multi-layered treatment regimen tailored to the physiological requirements of the target area.

Thus, when a smaller, more concentrated distribution is required, the catheter tip can be retracted to expose only a proximal zone with smaller, densely arranged micropores, which allows for a controlled and localized infusion. Conversely, for broader or more dispersed distribution, the catheter tip can be extended to progressively expose additional zones, such as zones with medium or larger-sized micropores and lower density. This gradual exposure of zones with different micropore sizes and configurations enables customization of the distribution shape, tailoring it to the specific therapeutic needs of the tissue environment. By adjusting the extent to which the distal tip is extended, the catheter design provides predictable and programmable control over the CED distribution, offering precision in targeting specific tissue areas while optimizing drug delivery.

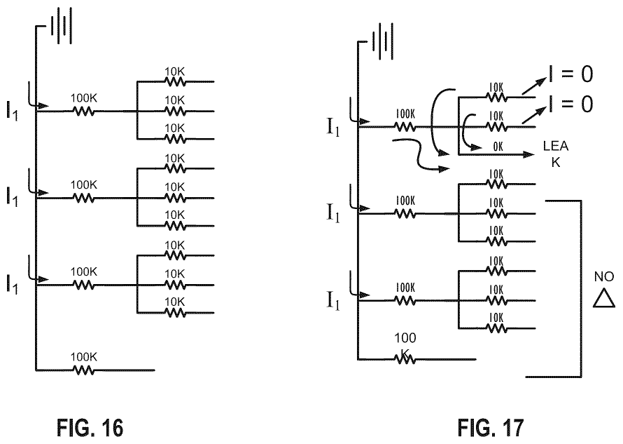

FIGS. 16 and 17 employ electrical circuit analogies to conceptualize how the resistance of each micropore 1102 in the distal tip portion 1110 can be engineered to dominate over the variable resistance of the surrounding tissue. In the diagram of FIG. 16, each branch of the circuit represents a flow path through a respective zone or set of micropores 1102. The upstream resistor with a relatively large impedance (labelled “100 K”) symbolizes the inherent hydraulic resistance (pressure) of a micropore or micropore zone, while the downstream network of smaller resistors (“10K” branches) represents the resistance of the biological tissue at multiple potential leakage sites around the catheter tip. Because the micropore resistance is deliberately made large relative to the tissue resistance, e.g., by making the micropore diameter small, the total flow in each branch (analogous to the infusion current I₁) is determined primarily by the fixed micropore resistance rather than by the variable tissue resistance downstream. Thus, even if the resistance of the tissue changes due to local compliance or the presence of a sulcus or vessel, the volumetric flow through each micropore remains substantially constant.

FIG. 17 illustrates the same concept when one branch experiences a loss of resistance (or pressure), analogous to a leakage path opening adjacent to a particular micropore. In this diagram a downstream resistor is shorted to ground (labelled “0K, LEAK”), resulting in zero resistance along that branch. Because the branches are parallel, the low-resistance path may carry almost all of the flow allocated to that branch (I=0 in the other downstream paths), but the flow through the other branches is not significantly altered. This demonstrates that, in a micropore-based catheter 1100 according to the present disclosure, leakage or a low-pressure zone at one micropore does not cause a pressure drop or loss of pressure or flow at the remaining micropores; each micropore or zone continues to deliver agent at its set flow rate. By analogy, increasing the pore diameter or density within a zone may correspond to decreasing the upstream resistance in that branch, thereby permitting a higher flow for that zone, while adding barriers between zones corresponds to electrically isolating the branches so that current (flow) does not redistribute. Similarly, adding additional infusion lines to separate zones is akin to supplying each branch with its own current source, allowing independent control of pressure and flow rate.

These circuit analogies reinforce the design principles embodied in the microporous distal tip portion 1110. By one or more of: selecting micropore diameters between about 1 μm and about 100 μm (e.g., 10-40 μm), arranging the micropores in discrete zones with independent infusion channels, providing the ability to vary pore size, density, and activation, the catheter 1100 can ensure that the resistance to flow is defined by the micropores 1102 rather than by unpredictable tissue heterogeneity. Whether the distal tip portion 1110 is extended or retracted, whether zones are pressurized independently, or whether micro-valves are opened or closed, the flow through each active micropore or zone may remain predictable and immune to the formation of low-pressure conduits in the tissue.

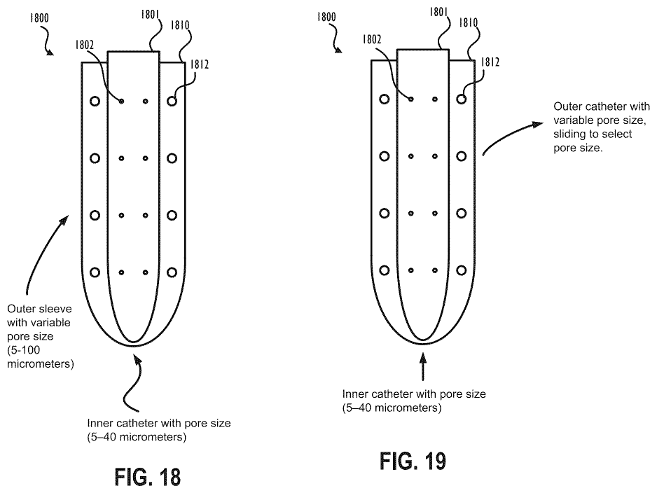

FIGS. 18 and 19 depict embodiments of a dual-layer microporous catheter 1800 in which an inner tube 1801 having a first set of micropores 1802 may be disposed concentrically within an outer tube 1810 having a second set of micropores 1412. In some embodiments, the inner tube 1800 may correspond to the capillary tube 230 described above and terminate in the distal tip portion 1110 that carries a circumferential array of fixed size micropores 1802. In other embodiments, the outer tube 1810 may correspond to the capillary tube 230, and the inner tube 1400 may be provided inside the distal tip portion 1110 of the capillary tube 230.

In either embodiment, the inner micropores 1802 may each have a hydraulic diameter in a range of about 1 μm to about 40 μm, and in certain embodiments between about 5 μm and about 40 μm, to provide a high, uniform flow resistance. Surrounding the inner tube may be the outer tube 1810 that forms an outer lumen and includes larger or variable sized micropores or apertures 1812 distributed along its outer circumferential surface.

As illustrated conceptually in FIGS. 18-19, the micropores or apertures 1812 in the outer tube 1810 may have diameters selected from a range that extends from approximately 5 μm up to 100 μm and may vary along the axial length or circumferential position of the sleeve. For example, the micropores 1812 may be distributed in subsets provided along different radial or axial sections of the tube 1810 and the outer tube 1810 may be radially spaced from the inner tube 1801 so that the outer micropores 1812 can be selectively aligned or misaligned with the inner micropores 1802. Also, as shown in FIGS. 18-19, distal ends of the inner and outer tubes 1801 and 1810 may be rounded or tapered to minimize tissue trauma during insertion.

In the embodiment of FIG. 18, the outer tube 1810 may be configured to rotate axially relative to the inner tube 1801, thereby modulating the effective pore size through which the agent exits. In the embodiment of FIG. 19, the outer tube 1810 may be configured to translate axially and optionally, also rotate relative to the inner tube 1801, thereby also modulating the effective pore size through which the agent exits. In either of FIGS. 18-19, when an outer micropores 1412 are aligned with inner micropores 1802, the two openings may cooperate to form a fluid connection that decreases the overall flow resistance and permits a flow at that location. Conversely, when the outer micropore or aperture 1812 is out of alignment with the inner micropores 1801, the outer tube 1810 may act to restrict the exposed area of the inner micropores 1801, increasing the resistance and thereby reducing or eliminating flow at the site of that outer micropore 1812.

Through controlled relative movement, achieved, for example, via a proximal control knob or motorized actuator, a clinician may select which inner or outer pores to activate and set their effective diameters or flow rates. In some embodiments the inner and outer layers may be connected to the same infusion line so that the total flow is distributed across the composite pores; in other embodiments they may define zones that are connected to separate infusion lines to allow distinct agents or pressures to be delivered through the different pore sets. The spacing and distribution density of the outer apertures can be tailored to match the distribution of inner micropores or intentionally offset to create staggered patterns. Optional seals or barriers between sections of the dual-layer tip can prevent fluid leakage into the annular space and maintain independent pressure zones as described previously.

This dual-layer design of FIGS. 18-19 may provide additional degrees of control beyond a single-layer microporous diffusion head. By selecting appropriate combinations of inner and outer pore sizes, and by dynamically adjusting their alignment, the physician can vary the hydraulic resistance of individual pores or zones in real time, producing infusion patterns that range from highly localized to broadly distributed. Moreover, the ability to connect zones of the inner tube 1801 and outer tube 1810 to the same or different infusion sources supports multiplexed delivery of therapeutic agents, and the integration of sensors and actuators (described above with respect to FIGS. 11-15) may be extended to the dual-layer configuration to enable closed loop control of infusion parameters.

In some embodiments, the placement of the microporous catheter can be facilitated by an active mechanical device rather than manual advancement with a stylet alone. FIG. 21 illustrates a scenario in which a microrobot 2105 is coupled to the distal end of the catheter 2100 and is used to pull the flexible, microporous catheter 2100 along a nonlinear trajectory through tissue 2110 toward a predetermined target site. The microrobot 2105 may incorporate locomotion components such as mechanically or magnetically actuated elements that allow it to steer through the interstitial matrix. As the microrobot 2105 advances, the catheter 2100 shaft may bend to follow the microrobot's 2105 path while the microporous distal tip portion with its array of micropores trails behind. Once the microrobot has reached the desired location, the stylet 240 (if present) can be retracted from the first lumen to impart flexibility, and infusion can commence through the capillary tube 230 and the exposed micropores 1102. Throughout the process, any sensors integrated into the catheter (e.g., pressure sensors, chemical sensors or positional sensors) may remain operational to monitor tissue conditions and configure infusion parameters.

FIG. 22 depicts two conceptual examples of microrobot-guided catheter insertion. In the top panel, the microrobot 2105 is shown dragging a microporous catheter 2100 along a sinusoidal path; the distal tip portion 1110 of the catheter 2100 may include multiple micropores 1102 distributed along its length. The microrobot 2105 may be mechanically or magnetically actuated to follow the prescribed trajectory, and the catheter's flexible body may conform to the path without kinking. In the bottom panel, the microrobot 2105 has negotiated a tight turn, drawing the microporous catheter into a curved configuration; the micropores 1102 remain oriented toward the tissue, ready to deliver the agent once the microrobot 2105 assisted catheter 2100 placement has completed. This approach may allow the catheter 2100 to be advanced along straight or curved paths, including complex three-dimensional trajectories, while maintaining precise control over the location and orientation of the microporous infusion head 1110. In certain embodiments, the microrobot 2105 may be tethered to the catheter by a mechanical linkage or may include a docking interface that engages the distal tip portion 1110 during advancement and that is adapted to disengage under predetermined conditions. Because the microrobot 2105 can traverse linear or nonlinear paths with high precision, it may be particularly suited for navigating around critical structures or through narrow anatomical corridors, ensuring that the microporous catheter 2100 can reach target sites that might otherwise be inaccessible or require more invasive approaches.

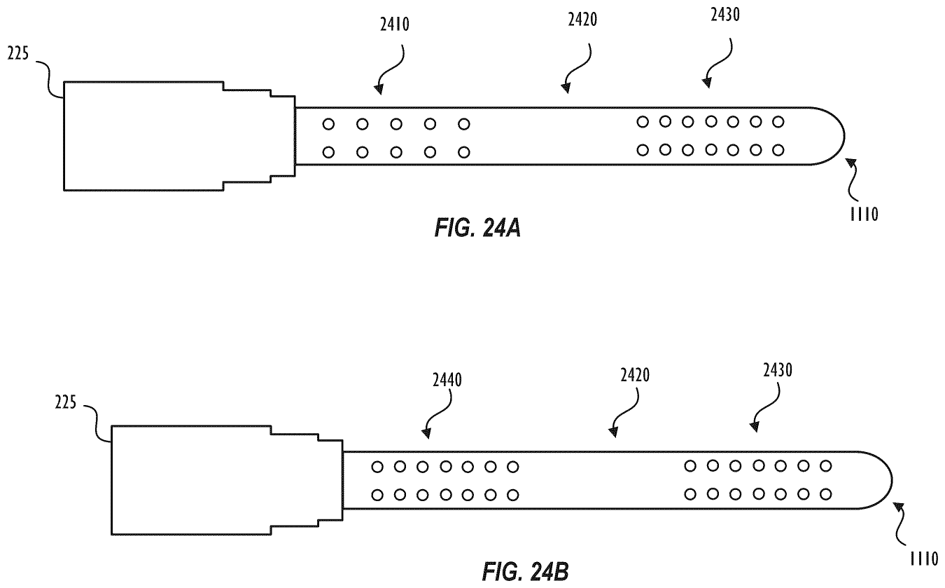

In some embodiments, the distal tip portion 1110 of the capillary tube may be subdivided into a sequence of discrete regions or zones having different pore characteristics, as shown in FIGS. 24A and 24B. FIGS. 24A-24B both show that the tip portion 1110 extends distally from the reinforcement sleeve 225 and is segmented into multiple zones (e.g., 2410, 2420, 2430, 2440) along its length. In FIG. 24A, a proximal zone 2410 adjacent the sleeve carries a relatively low density of micropores, providing a restricted number of flow pathways and therefore higher local flow resistance. This zone 2410 transitions into an intermediate region 2420 in which no micropores are formed; the absence of openings in this region creates a “gap” that separates adjacent pore clusters. Distal to the gap 2420 is a third zone 2430 having a higher pore density, in which the array of micropores is more closely spaced. In another embodiment as shown in FIG. 24B, the first region distal to the sleeve, labelled 2440, also has a high pore density similar to the distal zone 2430, separated by an intermediate, non-perforated region 2420. Although two and three-zone examples are depicted in FIGS. 24A-24B, the microporous tip portion 1110 may be divided into any number of regions, and the number, length and arrangement of zones can vary.

By combining zones (e.g., 2410, 2420, 2430, 2440) with different pore densities and interspersing them with one or more no-pore regions, the catheter 1100 can produce drug distribution profiles that differ from those achievable with end-port catheters. For example, the low-density zone 2410 of FIG. 24A may initiate infusion at a high resistance, while the high-density zone 2430 may promote a broader, lower-resistance distribution once upstream static pressure is overcome; and the intervening gap 2420 may delay infusion into the distal zone until the proximal zone has partially filled, thereby shaping the overall infusion volume. In FIG. 24B, two high-density zones 2440 and 2430 separated by a gap 2420 may allow symmetric or bilobed distributions. As described above with respect to other embodiments (e.g., in FIGS. 13-14), individual zones can be supplied by independent micro-infusion channels and fluidically isolated by internal barriers, permitting separate pressurization or flow-rate control; zones (e.g., 2410, 2420, 2430, 2440) may also differ in pore diameter, shape, distribution density or axial length. In addition, one or more zones may omit micropores entirely, as shown for region 2420, creating a discontinuity that may help tailor the spatial pattern of convection. The number of zones, their order along the tip, and their individual characteristics can be selected to match a desired three-dimensional distribution shape.

The gap region 2420 need not be an inert spacer; in some embodiments, it may incorporate features that further shape infusion or reduce reflux. For example, an outer surface (in whole or in part) of the no-pore region 2420 may have a stepped or tapered profile, a change in outer diameter, or a textured surface with microscopic ridges or dimples that enhance sealing against tissue and impede backward flow. Alternatively or additionally, the surface chemistry of the gap region 2420 can be modified with hydrophilic or hydrophobic coatings, hydrogels or lipid layers to adjust adhesion and wettability. These physical and physicochemical modifications may alter how interstitial fluid interacts with the catheter surface in a region of the zone 2420, providing another degree of control over fluid distribution without introducing additional pores. Such features can be employed alone or in combination with variable-density pore zones to tune the shape of the infusion cloud.

Because different therapeutic targets, such as irregularly shaped tumors or diffuse neurological structures, may require different infusion geometries, the distal tip portion 1110 can be manufactured or selected with zone configurations tailored to the target anatomy. A library of microporous tips may be provided, each with a distinct arrangement of zones differing in pore size, pore density, zone length or gap spacing. Alternatively, a patient-specific tip design can be created by modelling the desired distribution profile and translating it into a corresponding sequence of high-density, low-density and no-pore regions. When used together with the adjustable-length and zonal control features described above, such designs may enable programmable, predictable convection-enhanced delivery matched to the three-dimensional geometry of the tissue being treated.

### Example Method of Delivering a Therapeutic Agent Using Micropore Catheter

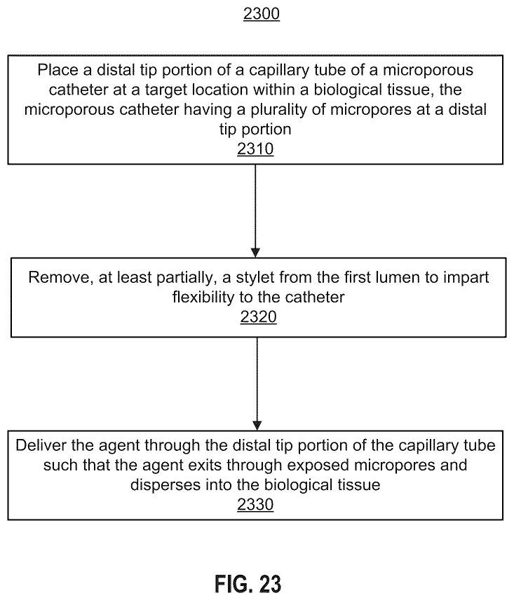

FIG. 23 is a flow chart illustrating a process 2300 for delivering a therapeutic agent to brain tissue using a micropore catheter (e.g., FIGS. 11-22), in accordance with one or more embodiments. The method 2300 begins with an operator placing 2310 a distal tip portion 1110 of a capillary tube 230 of a catheter (e.g., catheter 1100, 1800, 2100 of FIGS. 11-22) at a predetermined target location within a biological tissue.

To perform this step, the catheter may be inserted along a trajectory defined by stereotactic coordinates or image guidance. During insertion, the first lumen of the catheter may house a stylet that stiffens the multi-lumen tube 205 and provides the pushability needed to traverse soft tissues. Alternatively, or in addition, a microrobot 2105 as described in connection with FIGS. 21-22 may be attached to the distal end of the catheter and pull the catheter along a linear or curvilinear path, allowing the flexible, microporous segment to trail behind. The distal tip portion 1110 may protrude beyond the coupler 220 and reinforcement sleeve 225 of the catheter 1100, and the array of micropores 1102 formed therein may be configured to face outward toward the surrounding parenchyma. Before infusion, the operator may adjust the exposed length of the distal tip portion 1110 by telescoping it relative to the distal end of the multi-lumen tube, extending it to expose additional zones of micropores or retracting it to concentrate delivery. This length adjustment can be used to tailor the shape of distribution or the volume of tissue addressed during the procedure.

At step 2320, once the distal tip portion 1110 is correctly positioned, the stylet (e.g., stylet 240) may be withdrawn at least partially from the first lumen. Removal of the stylet may convert the previously rigid delivery system into a compliant assembly that can accommodate subtle tissue movements during infusion. The first lumen remains sealed at its distal end, preventing the migration of air into the target tissue. If the catheter incorporates an inner tube and outer sleeve at the distal tip, this step may also include rotating or translating the inner tube relative to the outer sleeve to align or misalign their respective micropores (e.g., micropores 1802 of the inner tube and apertures/micropores 1812 of the outer sleeve in FIGS. 18-19) and thereby adjust the effective pore size through which the agent will flow. Where zones of micropores are provided along the distal tip portion 1110, the clinician may open or close micro-valves associated with individual micropores, or selectively actuate micropore sets in different zones, to establish which pores will participate in the subsequent infusion.

At step 2330, the therapeutic agent may be delivered through the capillary tube 230 so that the agent emerges from the exposed micropores and disperses into the tissue. The infusion pump connected to the proximal end of the capillary tube may be actuated to create a controlled pressure gradient, and the infused agent may pass through the lumen, through the distal tip portion 1110 and out of micropores 1102. If the distal tip portion is divided into discrete zones, each zone may be supplied by an independent micro-infusion channel and pressurized separately; this may allow the operator to adjust flow rates among the zones to achieve a predetermined distribution profile.

In embodiments employing concentric inner and outer tubes, the alignment of the inner micropores and outer apertures set during step 2320 may remain fixed during infusion, ensuring that the effective pore sizes correspond to the desired flow resistance. The infusion may also entail concurrent or sequential delivery of different therapeutic agents through different zones, for example administering one agent through a proximal zone and a second agent through a distal zone, or varying dosages between zones. Throughout the infusion, any integrated sensors positioned near the distal tip portion 1110, such as pressure transducers, temperature probes, chemical concentration sensors or impedance electrodes, can monitor local tissue conditions and agent distribution. Data from these sensors may be fed to a control unit that adjusts infusion parameters such as flow rate, pressure, and zone selection in real time to optimize delivery and minimize reflux or leakage. Upon completion of the infusion, the catheter may be retracted along its insertion path, with the microrobot (e.g., 2105) or stylet (e.g., 240) again available to assist withdrawal if needed.

### Additional Configuration Considerations

The foregoing description of the embodiments has been presented for the purpose of illustration; it is not intended to be exhaustive or to limit the patent rights to the precise forms disclosed. Persons skilled in the relevant art can appreciate that many modifications and variations are possible in light of the above disclosure.

At least one embodiment is disclosed and variations, combinations, and/or modifications of the embodiment(s) and/or features of the embodiment(s) made by a person having ordinary skill in the art are within the scope of the disclosure. Alternative embodiments that result from combining, integrating, and/or omitting features of the embodiment(s) are also within the scope of the disclosure. Where numerical ranges or limitations are expressly stated, such express ranges or limitations may be understood to include iterative ranges or limitations of like magnitude falling within the expressly stated ranges or limitations (e.g., from about 1 to about 10 includes, 2, 3, 4, etc.; greater than 0.10 includes 0.11, 0.12, 0.13, etc.). The use of the term “about” means ±10% of the subsequent number, unless otherwise stated.

Use of the term “optionally” with respect to any element of a claim means that the element is required, or alternatively, the element is not required, both alternatives being within the scope of the claim. Use of broader terms such as comprises, includes, and having may be understood to provide support for narrower terms such as consisting of, consisting essentially of, and comprised substantially of. Accordingly, the scope of protection is not limited by the description set out above but is defined by the claims that follow, that scope including all equivalents of the subject matter of the claims. Each and every claim is incorporated as further disclosure into the specification and the claims are embodiment(s) of the present disclosure.

While several embodiments have been provided in the present disclosure, it should be understood that the disclosed systems and methods might be embodied in many other specific forms without departing from the spirit or scope of the present disclosure. The present examples are to be considered as illustrative and not restrictive, and the intention is not to be limited to the details given herein. For example, the various elements or components may be combined or integrated in another system or certain features may be omitted, or not implemented.

In addition, techniques, systems, subsystems, and methods described and illustrated in the various embodiments as discrete or separate may be combined or integrated with other systems, modules, techniques, or methods without departing from the scope of the present disclosure. Other items shown or discussed as coupled or directly coupled or communicating with each other may be indirectly coupled or communicating through some interface, device, or intermediate component whether electrically, mechanically, or otherwise.

Some portions of this description describe the embodiments in terms of algorithms and symbolic representations of operations on information. These algorithmic descriptions and representations are commonly used by those skilled in the data processing arts to convey the substance of their work effectively to others skilled in the art. These operations, while described functionally, computationally, or logically, are understood to be implemented by computer programs or equivalent electrical circuits, microcode, or the like.

Furthermore, it has also proven convenient at times, to refer to these arrangements of operations as modules, without loss of generality. The described operations and their associated modules may be embodied in software, firmware, hardware, or any combinations thereof.

Embodiments may also relate to an apparatus for performing the operations herein. This apparatus may be specially constructed for the required purposes, and/or it may comprise a general-purpose computing device selectively activated or reconfigured by a computer program stored in the computer. Such a computer program may be stored in a non-transitory, tangible computer readable storage medium, or any type of media suitable for storing electronic instructions, which may be coupled to a computer system bus. Furthermore, any computing systems referred to in the specification may include a single processor or may be architectures employing multiple processor designs for increased computing capability.

Finally, the language used in the specification has been principally selected for readability and instructional purposes, and it may not have been selected to delineate or circumscribe the patent rights. Many other embodiments will be apparent to those of skill in the art upon reviewing the above description. It is therefore intended that the scope of the patent rights be limited not by this detailed description, but rather by any claims that issue on an application based hereon. Accordingly, the disclosure of the embodiments is intended to be illustrative, but not limiting, of the scope of the patent rights, which is set forth in the following claims.

What is claimed is:

1. A medical catheter, comprising:
a multi-lumen tube comprising a proximal end, a distal end, a first lumen, and a second lumen that is independent of the first lumen, the first lumen being sealed at the distal end;
a capillary tube of the second lumen of the multi-lumen tube, the capillary tube comprising a distal tip portion for releasing an agent from the distal tip portion to a biological tissue, wherein the distal tip portion of the capillary tube is provided on an exposed portion of the capillary tube that protrudes beyond the distal end of the multi-lumen tube; and
a plurality of micropores formed in the distal tip portion of the capillary tube, each micropore having a diameter in a range of 1 to 100 micrometers.

2. The medical catheter of claim 1, wherein each of the plurality of micropores has a diameter in a range of 10 to 40 micrometers.

3. The medical catheter of claim 1, wherein the plurality of micropores are distributed along the distal tip portion circumferentially with a variable spacing, density or diameter to achieve predetermined infusion profiles.

4. The medical catheter of claim 1, wherein the plurality of micropores are grouped into a plurality of discrete zones along the distal tip portion, each zone having micropores of a different average diameter or distribution density from an adjacent zone.

5. The medical catheter of claim 4, further comprising:
a plurality of separate infusion channels within the capillary tube, each separate infusion channel fluidly coupled to a respective one of the plurality of discrete zones to permit independent control of infusion pressure or flow rate for each zone.

6. The medical catheter of claim 4, further comprising:
at least one barrier disposed between adjacent zones in the distal tip portion to fluidly isolate the plurality of discrete zones from one another.

7. The medical catheter of claim 1, wherein the exposed portion including the distal tip portion is configured to extend or retract relative to the distal end of the multi-lumen tube to selectively expose more or fewer micropores, wherein the distal tip portion has an adjustable length in a range of 5 millimeters to 50 millimeters.

8. The medical catheter of claim 1, further comprising:
an inner tube provided within the capillary tube at the distal tip portion, wherein the inner tube has a plurality of inner micropores of a first size, and the plurality of micropores formed along the distal tip portion of the capillary tube are a plurality of outer micropores that have a second variable size larger than the first size; and
an actuation mechanism adapted for relative rotation or axial translation between the inner tube and the distal tip portion of the capillary tube to align or misalign the inner and outer micropores relative to each other to modify an effective pore size through which the agent is infused.

9. The medical catheter of claim 1, further comprising:
at least one sensor coupled to the distal tip portion, the at least one sensor configured to detect one or more of tissue pressure, agent reflux, agent distribution, temperature, electrical impedance, or chemical concentration; and
a control unit configured to adjust infusion parameters based on data obtained from the at least one sensor.

10. The medical catheter of claim 1, further comprising:
a mechanical actuator or microrobot configured to advance the distal tip portion along a straight or curved path within the biological tissue.

11. A convection-enhanced delivery (CED) method, comprising:
placing a distal tip portion of a capillary tube of a catheter at a target location within a biological tissue, the catheter comprising:
a multi-lumen tube comprising a proximal end, a distal end, a first lumen, and a second lumen that is independent of the first lumen, the first lumen being sealed at the distal end;
the capillary tube of the second lumen of the multi-lumen tube, the capillary tube comprising the distal tip portion for releasing an agent from the distal tip portion to the biological tissue, wherein the distal tip portion of the capillary tube is provided on an exposed portion of the capillary tube that protrudes beyond the distal end of the multi-lumen tube; and
a plurality of micropores formed in the distal tip portion of the capillary tube, each micropore having a diameter in a range of 1 to 100 micrometers;
removing, at least partially, a stylet from the first lumen to impart flexibility to the catheter; and
delivering the agent through the distal tip portion of the capillary tube such that the agent exits through exposed micropores and disperses into the biological tissue.

12. The method of claim 11, further comprising:
prior to delivering the agent, adjusting a length of the distal tip portion of the exposed portion relative to the distal end of the multi-lumen tube to selectively expose more or fewer of the plurality of micropores.

13. The method of claim 11, wherein the plurality of micropores are grouped into a plurality of discrete zones along the distal tip portion, and delivering the agent further comprises:
independently pressurizing the discrete zones to achieve a predetermined distribution profile.

14. The method of claim 11, wherein the catheter includes an inner tube provided within the capillary tube at the distal tip portion, the inner tube having a plurality of inner micropores of a first size, and the plurality of micropores formed along the distal tip portion of the capillary tube being a plurality of outer micropores that have a second variable size larger than the first size, and wherein the method further comprises:
prior to delivering the agent, rotating or translating the inner tube relative to the distal tip portion of the capillary tube to align or misalign the inner and outer micropores relative to each other to modify an effective pore size through which the agent is delivered.

15. The method of claim 11, further comprising:
detecting one or more properties at the distal tip portion using at least one of a pressure sensor, a temperature sensor, a chemical sensor, an electrical impedance sensor, and a reflux detector; and
adjusting at least one infusion parameter based on the detected one or more properties.

16. The method of claim 11, further comprising:
advancing the distal tip portion along a straight or curved path within the biological tissue using a stylet or a microrobot.

17. The method of claim 11, wherein the plurality of micropores are grouped into discrete zones along the distal tip portion, and wherein delivering the agent further comprises:
infusing different therapeutic agents concurrently or sequentially through different ones of the discrete zones.

18. A catheter, comprising:
a multi-lumen tube comprising a proximal end, a distal end, a first lumen, and a second lumen that is independent of the first lumen, the first lumen being sealed at the distal end;
a capillary tube of the second lumen of the multi-lumen tube, the capillary tube comprising a distal tip portion for releasing an agent from the distal tip portion to a biological tissue, wherein the distal tip portion of the capillary tube is an exposed portion of the capillary tube that protrudes beyond the distal end of the multi-lumen tube; and
a plurality of micropores formed in the distal tip portion of the capillary tube.

19. The catheter of claim 18, wherein each of the plurality of micropores has a diameter in a range of 10 to 40 micrometers.

20. The catheter of claim 18, wherein the plurality of micropores are grouped into a plurality of discrete zones along the distal tip portion, each zone having micropores of a different average diameter or distribution density from an adjacent zone.
